SEQUENCE LISTING (34 sequences)

SEQ ID NO: 1 - DNA, 24 bp, Artificial Sequence
  Description of Artificial Sequence Primer
cattctggaaatgacagtgaagca

SEQ ID NO: 2 - DNA, 17 bp, Artificial Sequence
  Description of Artificial Sequence Primer
cagcgcagccgtcatct

SEQ ID NO: 3 - DNA, 20 bp, Artificial Sequence
  Description of Artificial Sequence Probe
ctccggaacctgcagcgggc

SEQ ID NO: 4 - DNA, 20 bp, Artificial Sequence
  Description of Artificial Sequence Primer
ttccctcgccactgctatct

SEQ ID NO: 5 - DNA, 18 bp, Artificial Sequence
  Description of Artificial Sequence Primer
gactcgcccgtggtgttg

SEQ ID NO: 6 - DNA, 23 bp, Artificial Sequence
  Description of Artificial Sequence Probe
cccaacaacgagaaaggccggaa

SEQ ID NO: 7 - DNA, 19 bp, Artificial Sequence
  Description of Artificial Sequence Primer
tccagagtgccaccgatgt

SEQ ID NO: 8 - DNA, 20 bp, Artificial Sequence
  Description of Artificial Sequence Primer
tccaccggctcactcttcac

SEQ ID NO: 9 - DNA, 26 bp, Artificial Sequence
  Description of Artificial Sequence Probe
ctgccttcctaggtgctcttgcgtca

SEQ ID NO: 10 - DNA, 18 bp, Artificial Sequence
  Description of Artificial Sequence Primer
accctccgccgagactct

SEQ ID NO: 11 - DNA, 22 bp, Artificial Sequence
  Description of Artificial Sequence Primer
tcccagaaccaatcaggttagc

SEQ ID NO: 12 - DNA, 22 bp, Artificial Sequence
  Description of Artificial Sequence Probe
cctgtcccacaggttcacggcg

SEQ ID NO: 13 - DNA, 20 bp, Artificial Sequence
  Description of Artificial Sequence Primer
ttctttcgcgtatgcctcaa

SEQ ID NO: 14 - DNA, 19 bp, Artificial Sequence
  Description of Artificial Sequence Primer
catcaggcaggctgaagga

SEQ ID NO: 15 - DNA, 23 bp, Artificial Sequence
  Description of Artificial Sequence Probe
actaccaggccagcgtgtcaccg

SEQ ID NO: 16 - DNA, 18 bp, Artificial Sequence
  Description of Artificial Sequence Primer
cccgcacccaggatgtgt

SEQ ID NO: 17 - DNA, 21 bp, Artificial Sequence
  Description of Artificial Sequence Primer
caccccagttggtctcacaga

SEQ ID NO: 18 - DNA, 24 bp, Artificial Sequence
  Description of Artificial Sequence Probe
cacctgcaatgaaccctggcagtg

SEQ ID NO: 19 - DNA, 19 bp, Artificial Sequence
  Description of Artificial Sequence Primer
cagctggacgccaatgagt

SEQ ID NO: 20 - DNA, 21 bp, Artificial Sequence
  Description of Artificial Sequence Primer
gccaatcaggtttttgcaaga

SEQ ID NO: 21 - DNA, 25 bp, Artificial Sequence
  Description of Artificial Sequence Probe
agcattaaggcacggcttcccttca

SEQ ID NO: 22 - DNA, 20 bp, Artificial Sequence
  Description of Artificial Sequence Primer
gcttcagcgagtgcatgaac

SEQ ID NO: 23 - DNA, 21 bp, Artificial Sequence
  Description of Artificial Sequence Primer
ttgatctgggtcatgcagttg

SEQ ID NO: 24 - DNA, 22 bp, Artificial Sequence
  Description of Artificial Sequence Probe
tgacccgcttcctgtccacgtg

SEQ ID NO: 25 - DNA, 18 bp, Artificial Sequence
  Description of Artificial Sequence Primer
ccacaaagcagccttgca

SEQ ID NO: 26 - DNA, 22 bp, Artificial Sequence
  Description of Artificial Sequence Primer
agtaagagcaggcagcatagca

SEQ ID NO: 27 - DNA, 25 bp, Artificial Sequence
  Description of Artificial Sequence Probe
agagctccatcatgcctggctcagc

SEQ ID NO: 28 - DNA, 19 bp, Artificial Sequence
  Description of Artificial Sequence Primer
gagcccgaagcggactact

SEQ ID NO: 29 - DNA, 23 bp, Artificial Sequence
  Description of Artificial Sequence Primer
ggttttctcatagatggcgttgt

SEQ ID NO: 30 - DNA, 23 bp, Artificial Sequence
  Description of Artificial Sequence Primer
tgctaaagaggtcacccgcgtgc

SEQ ID NO: 31 - DNA, 23 bp, Artificial Sequence
  Description of Artificial Sequence Primer
agcaggagggagaattacggaac

SEQ ID NO: 32 - DNA, 18 bp, Artificial Sequence
  Description of Artificial Sequence Primer
ttctgtggcctgcttggg

SEQ ID NO: 33 - DNA, 20 bp, Artificial Sequence
  Description of Artificial Sequence Primer
ggctccgtctctcctaaccg

SEQ ID NO: 34 - DNA, 19 bp, Artificial Sequence
  Description of Artificial Sequence Primer
gggccatgaggaacatcag

(19) United States
(12) Patent Application Publication
(10) Pub. No.: US 2004/0005631 A1
(43) Pub. Date: Jan. 8, 2004

(54) Assay

(76) Inventors: Jonathan Robert Lamb, Edinburgh (GB); Gerard Francis Hoyne, Canberra (AU); Margaret Jane Dallman, London (GB); Brian Robert Champion, Cambridge (GB)

Correspondence Address: FROMMER LAWRENCE & HAUG, 745 FIFTH AVENUE- 10TH FL., NEW YORK, NY, 10151, US

(21) Appl. No.: 10/357,321

(22) Filed: Feb. 3, 2003

Related U.S. Application Data
(63) Continuation-in-part of application No. PCT/GB01/03503, filed on Aug. 3, 2001.

(30) Foreign Application Priority Data
Aug. 4, 2000 (GB) GB 0019242.7

(51) Int. Cl. G01N 33/53

(52) U.S. Cl. 435/7.1

(57) ABSTRACT

A method for monitoring the immune system comprising monitoring the Notch signalling pathway.

The present invention relates to an assay method for use in monitoring the immune system and particularly, but not exclusively, the reactivity of T-cells to a given antigen. The invention further relates to a screening assay for modulators of Notch signalling and to modulators identifiable by such an assay.

### BACKGROUND OF THE INVENTION

Notch signal transduction plays a critical role in cell fate determination in vertebrate and invertebrate tissues. Notch is expressed at many stages of Drosophila embryonic and larval development and in many different cells implying a wide range of functions including an important role in neurogenesis and in the differentiation of mesodermal and endodermal cells. There are at least four mammalian Notch genes (Notch-1, Notch-2, Notch-3 and Notch-4). Notch-1, which most closely resembles the proteins of invertebrates and lower vertebrates, is widely expressed and is essential for early development. Recent evidence suggests that Notch signalling contributes to lineage commitment of immature T-cells in the thymus.

During maturation in the thymus, T-cells acquire the ability to distinguish self-antigens from those that are non-self, a process termed “self tolerance” (von Boehmer). Tolerance to a non-self antigen, however, may be induced by immunisation under specific conditions with a peptide fragment comprising that antigen. In autoimmune diseases such as multiple sclerosis, rheumatoid arthritis or diabetes, there is a failure of the proper regulation of tolerance. Improved treatment methods for re-establishing tolerance are desirable for autoimmune diseases. Similarly in allergic conditions and for transplantation of an organ or tissue from a donor individual, induction of tolerance to particular foreign antigens or profiles of foreign antigens is desirable.

The expression on the cell surface of normal adult cells of the peripheral immune system of Notch and its ligands, Delta and Serrate, suggests a role for these proteins in T-cell acquired immunocompetence (Hoyne et al. (2000) Int. Immunol, 12:177-185). T-cells express Notch mRNA constitutively. Delta expression is limited to only a subset of T-cells in the peripheral lymphoid tissues. Serrate expression is restricted to a subset of antigen presenting cells (APCs). These observations reinforce the view that the Notch receptor ligand family continues to regulate cell fate decisions in the immune system beyond embryonic development (Ellisen) with Notch signalling playing a central role in the induction of peripheral unresponsiveness (tolerance or anergy), linked suppression and infectious tolerance.

Linked suppression occurs when an intact antigenic molecule is used for challenge immunisation and is characterised by cells being tolerised against, not only the target antigen, but also other, non-target regions of the antigenic molecule (Hoyne et al.). Infectious tolerance is a process whereby it is possible to generate a class of regulatory T-cells which are able to transmit antigen-specific tolerance to other neighbouring T-cells (Qin and WO98/20142). The functional activity of these cells can be mimicked by over-expression of a Notch ligand protein on their cell surfaces or on the surface of antigen presenting cells. In particular, regulatory T-cells can be generated by over-expression of a member of the Delta or Serrate family of Notch ligand proteins. Delta or Serrate induced T-cells specific to one antigenic epitope are also able to transfer tolerance to T-cells recognising other epitopes on the same or related antigens, a phenomenon termed “epitope spreading”.

Thus, as described in WO 98/20142, WO 00/36089 and WO 01/35990, manipulation of the Notch signalling pathway can be used in immunotherapy and in the prevention and/or treatment of T-cell mediated diseases. In particular, allergy, autoimmunity, graft rejection, tumour induced aberrations to the T-cell system and infectious diseases caused, for example, by Plasmodium species, Microfilariae, Helminths, Mycobacteria, HIV, Cytomegalovirus, Pseudomonas, Toxoplasma, Echinococcus, Haemophilus influenza type B, measles, Hepatitis C or Toxicara, may be targeted. Notch ligand expression also plays a role in cancer. Indeed, upregulated Notch ligand expression has been observed in some tumour cells. These tumour cells are capable of rendering T-cells unresponsive to restimulation with a specific antigen, thus providing a possible explanation of how tumour cells prevent normal T-cell responses. Downregulation of Notch signalling in vivo in T-cells may be used to prevent tumour cells from inducing immunotolerance in those T-cells that recognise tumour-specific antigens. In turn, this allows the T-cells to mount an immune response against the tumour cells (WO00/35990).

It would be of great interest to be able to monitor not only the efficiency of such treatments, but also that of other methods of immunotherapy. The ability, in any case, to predict and/or measure tolerance or lack thereof (i.e. the reactivity of the immune system, and in particular of T-cells) presents innumerable applications. The present invention seeks to provide such an assay.

### STATEMENTS OF INVENTION

According to a first aspect of the present invention, there is provided a method of monitoring the immune system comprising a step of detecting modulation of Notch signalling.

According to a further aspect of the present invention, there is provided a method for detecting or monitoring T-cell activation or inactivation comprising a step of detecting modulation of Notch signalling.

According to a further aspect of the present invention, there is provided a method for detecting or monitoring immunological tolerance or activity comprising a step of detecting modulation of Notch signalling.

According to another aspect of the present invention, there is provided a method for monitoring the efficacy of immunotherapy comprising a step of detecting modulation of Notch signalling.

According to yet another aspect of the present invention, there is provided a method for detecting or monitoring the reactivity of a T-cell to an antigen comprising a step of detecting modulation of Notch signalling.

The expressions “a T-cell”, “T-cell” and “T-cells” are used interchangeably herein and refer to a single T-cell or to a T-cell population.

As described above, the Notch signalling pathway plays an important role in the lineage commitment of immature T-cells. Measuring variations in the levels of signalling will therefore reflect changes in T-cell responsiveness. For example, while an increase in Notch signalling may reflect induced tolerance, a reduction in Notch signalling may reflect increased reactivity of a T-cell to a given antigen. As, such, according to a further aspect of the present invention, there is provided a method for detecting or monitoring Notch signalling comprising a step of detecting modulation of Notch signalling.

Detecting modulation of Notch signalling will preferably comprise a step of detecting cleavage of the cleavable intracellular domain of Notch; detecting interaction of Suppresser of Hairless (Su(H)), or homologues thereof, with Notch; detecting interaction of Deltex, or homologues thereof, with Notch; detecting interaction of Deltex, or homologues thereof, with Grb2; detecting interaction of Deltex, or homologues thereof, with Grb2; detecting modulation of the Ras-JnK signalling pathway; or detecting cleavage of the cleavable extracellular domain of Delta.

According to another aspect of the present invention, there is provided a method for detecting or monitoring the reactivity of a T-cell to an antigen comprising the steps of:

a. providing a T-cell in the presence of an antigen;

b. detecting modulation of Notch signalling within the T-cell; and

c. determining the reactivity of the T-cell to said antigen.

Step (b) may comprise a step of detecting cleavage of the cleavable intracellular domain of Notch. Alternatively, step (b) may comprise, a step of detecting interaction of Suppressor of Hairless (Su(H)), or homologues thereof, with Notch. Alternatively, step (b) may comprise a step of detecting interaction of Deltex, or homologues thereof, with Notch. Alternatively, step (b) may comprise a step of detecting interaction of Deltex, or homologues thereof, with Grb2. Alternatively, step (b) may comprise a step of detecting modulation of the Ras-JnK signalling pathway. Alternatively, step (b) may comprise a step of detecting cleavage of the cleavable extracellular domain of Delta.

In a preferred embodiment, the method of the invention comprises a further step of comparing detectable Notch signalling with a reference signal.

The step of detecting or monitoring modulation of Notch signalling may comprise a nucleic acid assay or a protein assay. Preferably, the protein assay will comprise detecting cleaved Notch intracellular domain. Alternatively, the protein assay will comprise detecting cleaved Notch extracellular domain. Alternatively, the protein assay will comprise detecting a cleaved Notch ligand extracellular domain, such as a Delta or Serrate extracellular domain. In a preferred embodiment of this aspect, the protein assay comprises detecting a cleaved Notch ligand extracellular domain.

In a preferred embodiment, the step of detecting or monitoring modulation of Notch signalling will comprise a step of detecting modulation of the expression of a target gene of Notch signalling. Target genes of Notch signalling may include, but are not limited to Deltex, Suppressor of Deltex, genes of the Hes family including Hes-1, E(spl) complex genes, Il-10, CD antigens such as CD-23 and CD-4, CTLA-4, Dll-1, Numb, members of the Fringe group, Mastermind, Dsh and dlx-1. Alternatively, the step of detecting or monitoring modulation of Notch signalling may comprise a step of detecting modulation of the expression of a Notch ligand or Notch receptor such as Delta 1-4, Serrate 1 or 2, Notch 1-4, and Su(H) or homologues, variants and derivatives thereof. In an even more preferred embodiment, the target gene is not IL-10 or a cytokine.

The method of the present invention can be used for detecting increased reactivity of a T-cell or T-cell population to an antigen. In a preferred embodiment, it will be used for detecting decreased reactivity of a T-cell or T-cell population to an antigen (i.e. T-cell tolerisation). The antigen may be a self or non-self (foreign) antigen.

According to a further aspect of the present invention, there is provided a method for detecting or monitoring the reactivity of a T-cell or T-cell population to an antigen comprising the steps of:

a. providing a ligand capable of binding to Notch such as to modulate Notch signalling;

b. providing a T-cell having a Notch receptor or a functional variant thereof capable of transducing Notch signalling;

c. allowing the ligand to bind to Notch in the presence of an antigen;

d. detecting modulation of Notch signalling; and

e. determining the reactivity of the T-cell to said antigen using a method of the invention.

According to a yet further aspect of the present invention, there is provided a method for detecting or monitoring Notch signalling comprising the steps of:

a. providing a ligand capable of binding to Notch such as to modulate Notch signalling;

b. providing a Notch receptor or a functional variant thereof;

c. allowing the ligand to bind to Notch; and

d. detecting modulation of Notch signalling using a method of the invention.

According to another aspect of the present invention, there is provided a kit for detecting or monitoring modulation of Notch signalling comprising a plurality of sequences or binding agents specific to nucleic acid or protein targets of Notch signalling. Preferably, the kit will be substantially free of unrelated sequences or binding agents.

According to a further aspect of the present invention, there is provided a nucleic acid or protein array consisting essentially of a plurality of sequences or binding agents specific to nucleic acid or protein targets of Notch signalling.

According to yet another aspect of the present invention, there is provided a method of screening for modulators of Notch signalling comprising the steps of:

a. providing a candidate modulator;

b. providing a Notch receptor or a functional variant thereof;

c. bringing Notch and the candidate modulator together under conditions such that the modulator interacts with Notch; and

d. detecting modulation of Notch signalling using a method of the invention.

According to yet another aspect of the present invention, there is provided a Notch ligand identifiable using the screening method of the invention.

According to a further aspect of the present invention, there is provided the use of a detectable signal of Notch signalling for detecting or monitoring the reactivity of a T-cell or T-cell population to an antigen.

According to a further aspect of the present invention, there is provided the use of a detectable signal of Notch signalling for monitoring the immune system.

According to a further aspect of the present invention, there is provided the use of a detectable signal of Notch signalling for detecting or monitoring T-cell activation or inactivation.

According to a yet further aspect of the present invention, there is provided the use of a detectable signal of Notch signalling for detecting or monitoring immunological tolerance or activation.

As indicated above, the ability to monitor the immune system in a simple and efficient assay presents innumerable applications. By way of illustration only, the possibility of measuring the efficacy of immunotherapy would be highly advantageous. Such an assay could, for example, be used to detect induced tolerance or anergy in patients being treated with drugs. For instance, transplant patients are prescribed drugs, such as cyclosporin, azathioprine, basiliximab or sirolimus, in an attempt to prevent graft rejection. It would be desirable to design a rationale for withdrawing such immunosuppressive drugs, such that patients would not be put at risk. An assay for measuring tolerance would enable patients to come off immunosuppressive medication in a more controlled and therefore less speculative manner.

In another example, allergy sufferers may benefit from an assay which would allow the responsiveness of their immune systems to be compared before and after therapy, such as allergy immunotherapy. Allergy immunotherapy typically involves administering increasing doses of an allergen (for example pollen allergens such as Phl p 1, House Dust Mite antigens such as Der p1 or Der f 1, dog and cat allergens such as Fel d 1 and Can f 1 or food allergens) over a period of several months or years with the aim of increasing tolerance over time. This present method of ensuring reduced sensitivity could remove the risk of previous tests involving exposure, even if only in small quantities, to an allergen which has the potential of causing inflammatory reactions or even anaphylactic shock. In addition, the present assay method would provide a much more objective measure of the effectiveness of therapy than the rather subjective symptom-based measures which are often used at present. Similarly, the ability to detect an immune response could be used in identifying the cause of an allergic reaction by monitoring the activity of the immune system in the presence of different potential allergens.

In an alternative scenario, the assay of the invention could be used to check for successful immunization against a given disease antigen. In one scenario, the assay method could be used to determine the state of a patient's immune system after administration of a vaccine, such that the degree of increase of immune system activity could indicate the effectiveness of the vaccination (a decrease in Notch signalling generally indicating increased vaccine effectiveness). As we described above, downregulation of Notch signalling in vivo in T-cells may be used to prevent tumour cells from inducing immunotolerance in those T-cells that recognise tumour-specific antigens. Thus, in an alternative scenario, it would be useful to be able to check for increased reactivity of T-cells and therefore for successful anti-tumour treatment.

Other treatments and/or diseases which could be monitored with the present assay method are described in more detail below.

### DETAILED DESCRIPTION

Various preferred features and embodiments of the present invention will now be described by way of non-limiting example and with reference to the accompanying drawings in which:

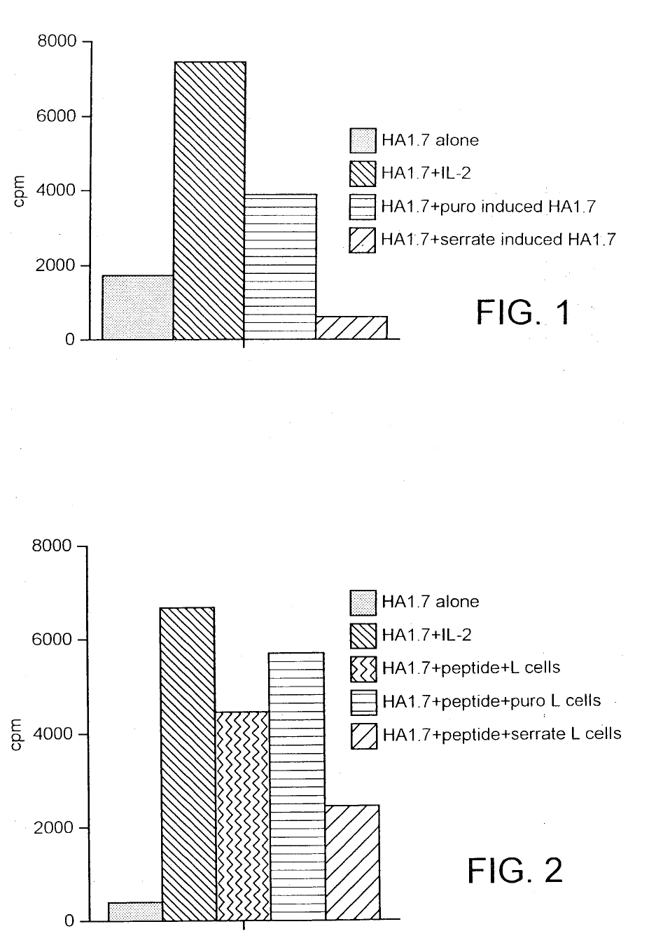

FIG. 1 shows the results of the experiment described in Example 1;

FIG. 2 shows the results of the experiment described in Example 2;

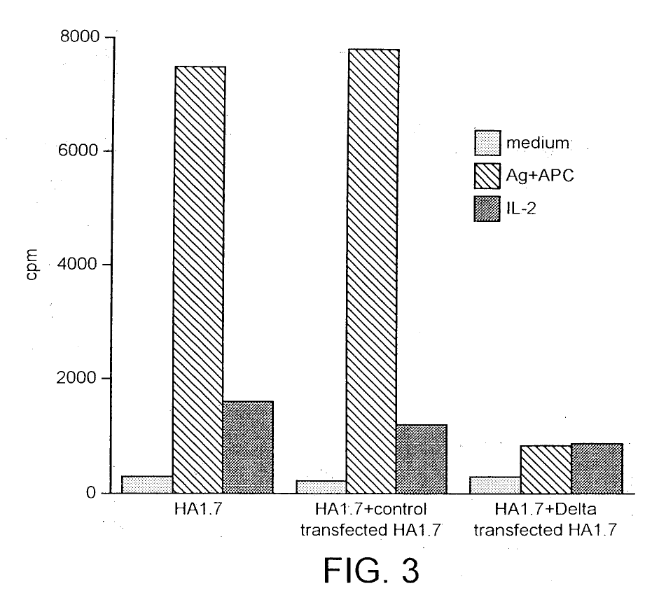

FIG. 3 shows the results of the experiment described in Example 3;

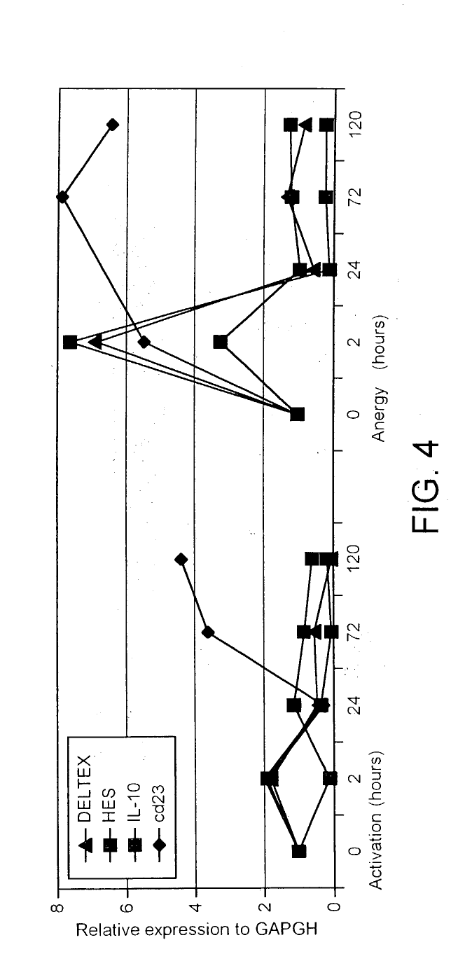

FIG. 4 shows the results of the experiment described in Example 4;

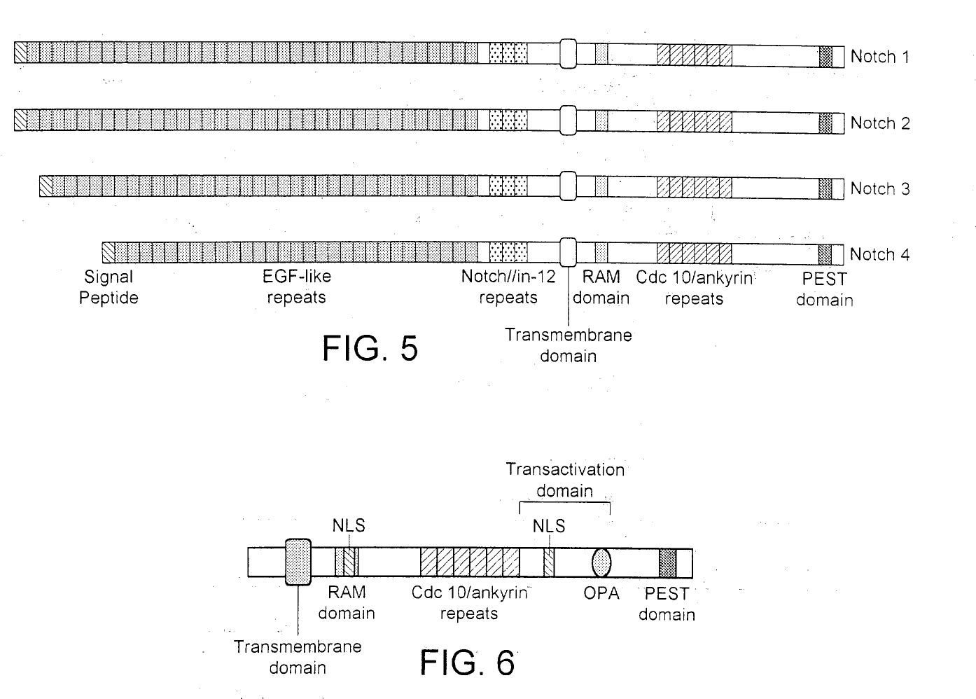

FIG. 5 shows a schematic representation of Notch 1-4;

FIG. 6 shows a schematic representation of Notch Intracellular Domain;

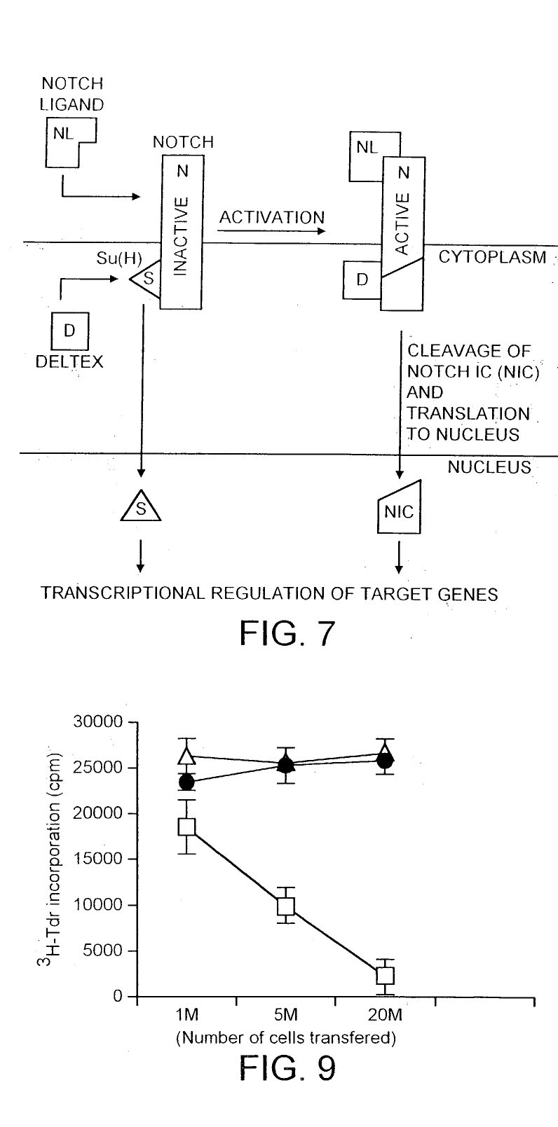

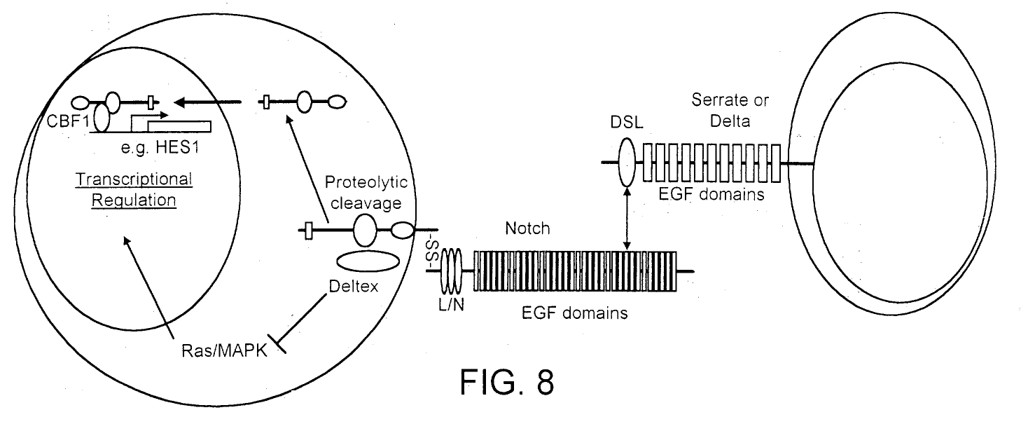

FIGS. 7 and 8 show schematic representations of the Notch signalling pathway;

FIG. 9 shows the results of the experiment described in Example 5;

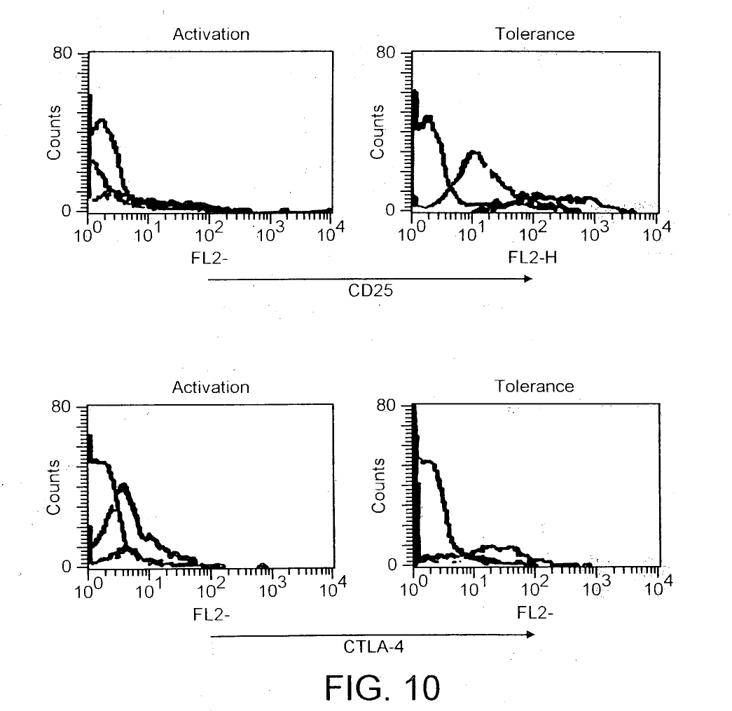

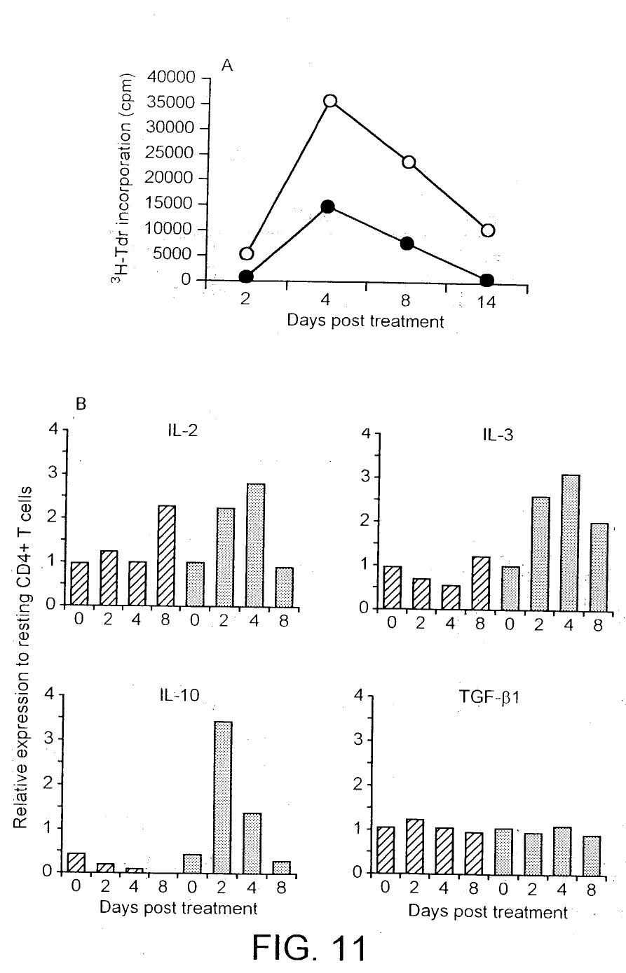

FIG. 10 shows the results of the experiment described in Example 6;

FIGS. 11A and 11B show the results of the experiment described in Example 7;

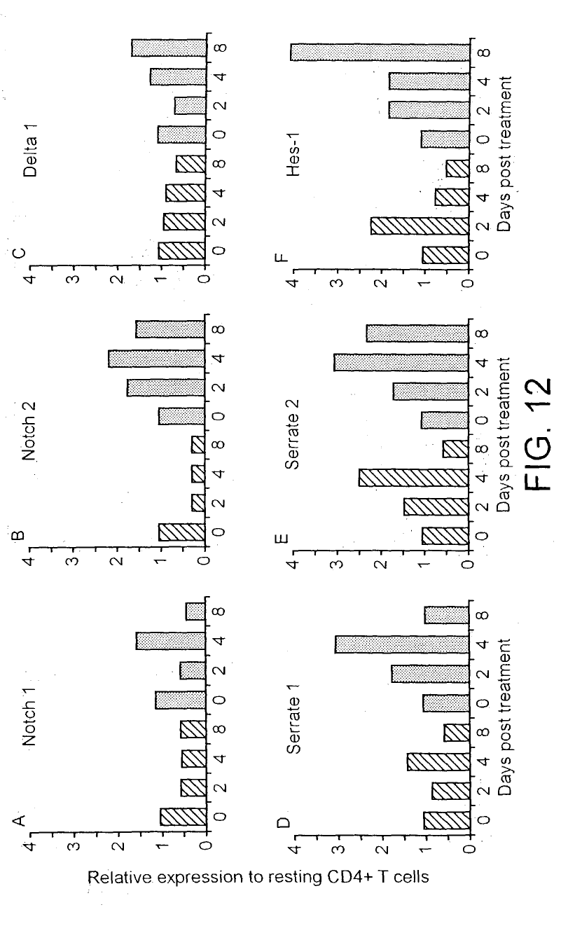

FIGS. 12 A-F shows the results of the experiment described in Example 8; and

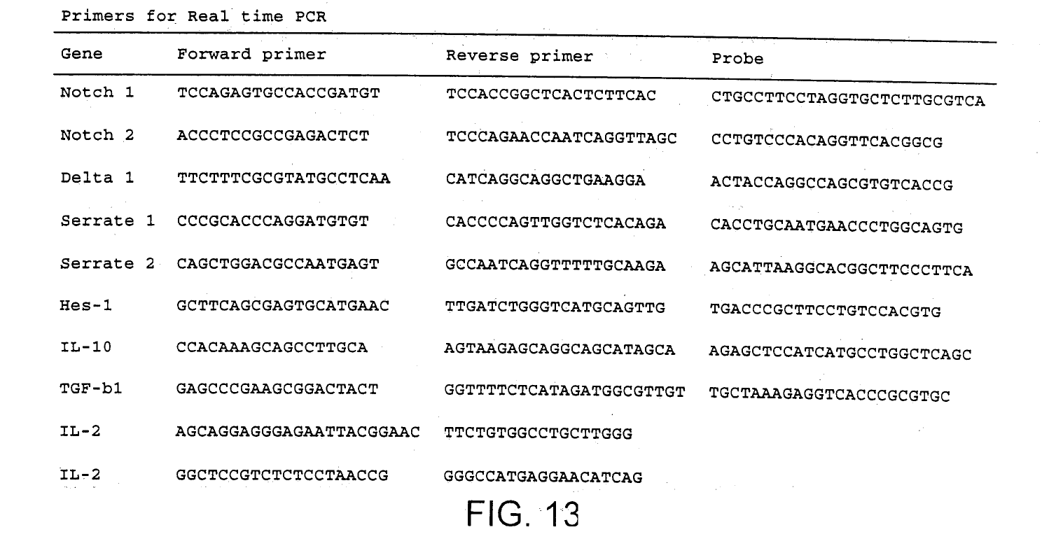

FIG. 13 shows examples of suitable primers and probes for use in the present invention.

Generally, the nomenclature used herein and the laboratory procedures in cell culture, molecular genetics, nucleic acid chemistry and hybridisation described below are those well known and commonly employed in the art. Standard techniques are used for recombinant nucleic acid methods, polynucleotide synthesis, and microbial culture and transformation (e.g. electroporation, lipofection). Generally, enzymatic reactions and purification steps are performed according to the manufacturer's specifications. The techniques and procedures are generally performed according to conventional methods in the art and various general references (see generally, Sambrook et al Molecular Cloning: A Laboratory Manual, 2^{nd} ed. (1989) Cold Spring Harbor Laboratory Press, Cold Spring Harbor, N.Y., which is incorporated herein by reference) which are provided throughout this document.

The present invention relates to an assay method for detecting or monitoring the reactivity of a T-cell or T-cell population to an antigen by detecting modulation of Notch signalling.

Notch Signalling

As used herein, the expression “Notch signalling” is synonymous with the expression “the Notch signalling pathway” and refers to any one or more of the upstream or downstream events that result in, or from, (and including) activation of the Notch receptor.

Preferably, by “Notch signalling” we refer to any event directly upstream or downstream of Notch receptor activation or inhibition including activation or inhibition of Notch/Notch ligand interactions, upregulation or downregulation of Notch or Notch ligand expression or activity and activation or inhibition of Notch signalling transduction including, for example, proteolytic cleavage of Notch and upregulation or downregulation of the Ras-Jnk signalling pathway.

Put another way, by “Notch signalling” we refer to the Notch signalling pathway as a signal tranducing pathway comprising elements which interact, genetically and/or molecularly, with the Notch receptor protein. For example, elements which interact with the Notch protein on both a molecular and genetic basis are, by way of example only, Delta, Serrate and Deltex. Elements which interact with the Notch protein genetically are, by way of example only, Mastermind, Hairless, Su(H) and Presenilin.

In one aspect, Notch signalling includes signalling events taking place extracellularly or at the cell membrane. In a further aspect, it includes signalling events taking place intracellularly, for example within the cell cytoplasm or within the cell nucleus.

A very important component of the Notch signalling pathway is Notch receptor/Notch ligand interaction. Thus the Notch signalling detected by the invention may involve detecting changes in expression, nature, amount or activity of Notch ligands or receptors or their resulting cleavage products. In addition, the Notch signalling detected by the invention may involve detecting changes in expression, nature, amount or activity of Notch signalling pathway G-proteins or Notch signalling pathway enzymes such as proteases, kinases (e.g. serine/threonine kinases), phosphatases, ligases (e.g. ubiquitin ligases) or glycosyltransferases. Alternatively the signalling detected by the invention may involve detecting changes in expression, nature, amount or activity of DNA binding elements such as transcription factors.

In a preferred form of the invention the signalling detected is specific signalling, meaning that the signal detected results substantially or at least predominantly from the Notch signalling pathway, and preferably from Notch/Notch ligand interaction, rather than any other significant interfering or competing cause. The Notch signalling pathway is described in more detail below.

Notch signalling directs binary cell fate decisions in the embryo. Notch was first described in Drosophila as a transmembrane protein that functions as a receptor for two different ligands, Delta and Serrate. Vertebrates express multiple Notch receptors and ligands. At least four Notch receptors (Notch-1, Notch-2, Notch-3 and Notch-4) have been identified to date in human cells.

Notch proteins are synthesized as single polypeptide precursors that undergo cleavage via a Furin-like convertase that yields two polypeptide chains that are further processed to form the mature receptor. The Notch receptor present in the plasma membrane comprises a heterodimer of two Notch proteolytic cleavage products, one comprising an N-terminal fragment consisting of a portion of the extracellular domain, the transmembrane domain and the intracellular domain, and the other comprising the majority of the extracellular domain. The proteolytic cleavage step of Notch to activate the receptor occurs and is mediated by a furin-like convertase.

Notch receptors are inserted into the membrane as disulphide-linked heterodimeric molecules consisting of an extracellular domain containing up to 36 epidermal growth factor (EGF)-like repeats and a transmembrane subunit that contains the cytoplasmic domain. The cytoplasmic domain of Notch contains six ankyrin-like repeats, a polyglutamine stretch (OPA) and a PEST sequence. A further domain termed RAM23 lies proximal to the ankyrin repeats and, like the ankyrin-like repeats, is involved in binding to a transcription factor, known as Suppressor of Hairless [Su(H)] in Drosophila and CBF1 in vertebrates (Tamura). The Notch ligands also display multiple EGF-like repeats in their extracellular domains together with a cysteine-rich DSL (Delta-Serrate Lag2) domain that is characteristic of all Notch ligands (Artavanis-Tsakonas).

The Notch receptor is activated by binding of extracellular ligands, such as Delta (Delta 1, 3 or 4), Serrate (Serrate 1 or 2 or their homologues Jagged 1 and 2) and Scabrous, to the EGF-like repeats of Notch's extracellular domain. Delta requires cleavage for activation. It is cleaved by the ADAM disintegrin metalloprotease Kuzbanian at the cell surface, the cleavage event releasing a soluble and active extracellular fragment of Delta. An oncogenic variant of the human Notch-1 protein, also known as TAN-1, which has a truncated extracellular domain, is constitutively active and has been found to be involved in T-cell lymphoblastic leukemias.

The cdc10/ankyrin intracellular-domain repeats mediate physical interaction with intracellular signal transduction proteins. Most notably, the cdc10/ankyrin repeats interact with Suppressor of Hairless [Su(H)]. Su(H) is the Drosophila homologue of C-promoter binding factor-1 [CBF-1], a mammalian DNA binding protein involved in the Epstein-Barr virus-induced immortalization of B-cells. It has been demonstrated that, at least in cultured cells, Su(H) associates with the cdc10/ankyrin repeats in the cytoplasm. Upon interaction of the Notch receptor with its ligand Delta on adjacent cells Su(H) disassociates from the Notch intracellular domain, where it is replaced by Deltex, and translocates into the nucleus. Su(H) includes responsive elements found in the promoters of several genes and has been found to be a critical downstream protein in the Notch signalling pathway. Target genes of Su(H) and of Notch signalling in general are listed below. The involvement of Su(H) in transcription is thought to be modulated by Hairless.

The intracellular domain of Notch (NotchIC) also has a direct nuclear function (Lieber). Recent studies have indeed shown that Notch activation requires that the six cdc10/ankyrin repeats of the Notch intracellular domain reach the nucleus and participate in transcriptional activation. The site of proteolytic cleavage on the intracellular tail of Notch has been identified between gly1743 and val1744 (termed site 3, or S3) (Schroeter). It is thought that the proteolytic cleavage step that releases the NotchIC for nuclear entry is dependent on Presenilin activity.

The intracellular domain has been shown to accumulate in the nucleus where it forms a transcriptional regulator complex with other transcription factors such as the CSL family member CBF1 (suppressor of hairless, Su(H) in Drosophila, Lag-2 in C. elegans) (Schroeter; Struhl) and Mastermind (MAML½). The NotchIC-CBF1 complexes then activate target genes, such as the bHLH proteins HES (hairy-enhancer of split like) 1 and 5 (Weinmaster). This nuclear function of Notch has also been shown for the mammalian Notch homologue (Lu).

NotchIC processing occurs only in response to binding of Notch ligands Delta or Serrate/Jagged. The post-translational modification of the nascent Notch receptor in the Golgi (Munro; Ju) appears, at least in part, to control which of the two types of ligand it interacts with on a cell surface. The Notch receptor is modified on its extracellular domain by Fringe, a glycosyl transferase enzyme that binds to the Notch/Lin motif. Fringe modifies Notch by adding O-linked fucose groups to the EGF-like repeats (Moloney; Bruckner). This modification by Fringe does not prevent ligand binding, but may influence ligand induced conformational changes in Notch. Furthermore, recent studies suggest that the action of Fringe modifies Notch to prevent it from interacting functionally with Serrate/Jagged ligands but allow it to preferentially interact with Delta (Panin; Hicks). Although Drosophila has a single Fringe gene, vertebrates are known to express multiple genes (Radical, Manic and Lunatic Fringes) (Irvine).

Thus, signal transduction from the Notch receptor can occur via different pathways (FIGS. 7 and 8). The better defined pathway involves proteolytic cleavage of the intracellular domain of Notch (NotchIC) that translocates to the nucleus and forms a transcriptional activator complex with the CSL family protein CBF1 (supressor of hairless, Su(H) in Drosophila, Lag-2 in C. elegans). NotchIC-CBF1 complexes then activate target genes, such as Deltex or the bHLH proteins HES (hairy-enhancer of split like) 1 and 5. Notch can also signal in a CBF1-independent manner that involves the cytoplasmic zinc finger containing protein Deltex (FIG. 8). Unlike CBF1, Deltex does not move to the nucleus following Notch activation. Instead, it interacts with Grb2 and modulates the Ras-Jnk signalling pathway which, in turn, modulates transcription of target genes.

The present invention is based on the realisation that knowledge of the Notch signalling pathway can be used in designing assay methods for monitoring modulation of the pathway and therefore for monitoring the reactivity of T-cells to a given antigen. Indeed, it will be appreciated that changes in the expression, activity, activation, interaction or physical state (e.g. cleavage or phosphorylation) of any one or more of the above described components of Notch signalling may be used to evaluate the degree of Notch signalling overall and therefore to establish the reactivity (or lack thereof) of a T-cell, or T-cell population, to a given antigen.

The term “modulate” as used herein refers to a change or alteration in the biological activity of the Notch signalling pathway or a target signalling pathway thereof. Thus, modulation of Notch signalling includes inhibition or down-regulation of Notch signalling, e.g. by compounds which block, at least to some extent, the normal biological activity of the Notch signalling pathway. Alternatively, the term “modulation” may refer to the activation or up-regulation of Notch signalling, e.g. by compounds which stimulate or upregulate, at least to some extent, the normal biological activity of the Notch signalling pathway.

Assay Methods

Notch signalling can be monitored either directly or indirectly, through protein assays or through nucleic acid assays, by detection of a signal. Such a “detectable signal” may be any detectable manifestation attributable to the modulation (i.e. increase or decrease) of Notch signalling, e.g. any detectable manifestation attributable to cleavage of the Notch intracellular domain; to the interaction, or lack of interaction, of Notch and Su(H); to the interaction, or lack of interaction, of Notch and Deltex; to the interaction, or lack of interaction, of Deltex and Grb2; to the modulation of the Ras-Jnk signalling pathway; or to cleavage of the Delta extracellular domain.

Thus, Notch signalling may conveniently be determined by detecting changes in the nature or amount of detectable signal (preferably, changes in levels of target nucleic acids or proteins) which are either components of the Notch signalling pathway or are specifically created or affected by Notch signalling.

Protein Assays

The advantage of using a protein assay is that Notch activation can be directly measured. Assay techniques that can be used to determine levels of a polypeptide are well known to those skilled in the art. Such assay methods may be, for example, competitive or non-competitive assay systems, membrane based, solution based or chip based and include radioimmunoassays, Western Blot analysis, antibody sandwich assays, antibody detection and ELISA assays.

In one embodiment, for example, a two-site monoclonal-based immunoassay utilising monoclonal antibodies reactive to two non-interfering epitopes on a polypeptide may be used. In another embodiment a competitive binding assay may be employed. These and other assays are described, for example, in Hampton et al., and Maddox et al.

Thus, the invention also provides diagnostic kits, comprising in one or more containers one or more binding agents or reagents for an appropriate marker or markers of the Notch signalling pathway. Such a kit may for example comprise one or more of the following: (1) instructions for using the binding agent or reagent for diagnosis, prognosis, therapeutic monitoring, drug development or any combination of these applications; (2) a labelled binding partner to the binding agent or reagent; and/or (3) a solid phase (such as a reagent strip) on which the binding agent or reagent may if desired be immobilized. If no labelled binding partner to the binding agent or reagent is provided, the binding agent or reagent itself may be labelled with a detectable moiety, such as a chemiluminescent, enzymatic, fluorescent, or radioactive moiety. A kit can optionally further comprise a predetermined amount of an isolated marker protein or a nucleic acid encoding a marker, e.g., for use as a standard or control.

The invention, in certain embodiments, includes antibodies specifically recognising and binding to polypeptides.

Antibodies may be recovered from the serum of immunised animals. Various procedures known in the art may be used for the production of polyclonal antibodies. In a particular embodiment, rabbit polyclonal antibodies to an epitope of a marker, or a subsequence therof, can be obtained. For the production of antibodies, various host animals can be immunized by injection with the native marker, or a synthetic version, or derivative (e.g., fragment) thereof, including but not limited to rabbits, mice, horses, goats, rats, chickens, etc. A variety of adjuvants may optionally be used to increase the immunological response if required, depending on the host species, and including but not limited to complete or incomplete Freund's Adjuvant, mineral gels such as aluminium hydroxide, surface active substances such as lysolecithin, pluronic polyols, polyanions, peptides, oil emulsions, keyhole limpit hemocyanins, dinitrophenol, and potentially useful human adjuvants such as BCG (bacillus Calmette-Guerin) and Corynebacterium parvum.

Monoclonal antibodies may be prepared from cells from immunised animals in the conventional manner. For preparation of monoclonal antibodies directed towards a marker sequence or analogue thereof, any technique which provides for the production of antibody molecules by continuous cell lines in culture may be used. For example, the hybridoma technique originally developed by Kohler and Milstein, as well as the trioma technique, the human B-cell hybridoma technique (Kozbor et al.), and the EBV-hybridoma technique to produce human monoclonal antibodies (Cole et al.) may be used.

Techniques for the production of single chain antibodies may also be used, as may techniques described for the construction of Fab expression libraries (Huse et al.) to allow rapid and easy identification of monoclonal Fab fragments with the desired specificity for markers, and their derivatives or analogues.

The antibodies of the invention are useful for identifying cells expressing the genes being monitored.

Antibodies according to the invention may be whole antibodies of natural classes, such as IgE and IgM antibodies, but are preferably IgG antibodies. Moreover, the invention includes antibody fragments, such as Fab, F(ab′)2, Fv and ScFv. Small fragments, such Fv and ScFv, possess advantageous properties for diagnostic and therapeutic applications on account of their small size and consequent superior tissue distribution.

The antibodies may comprise a label. Especially preferred are labels which allow the imaging of the antibody in neural cells in vivo. Such labels may be radioactive labels or radioopaque labels, such as metal particles, which are readily visualisable within tissues. Moreover, they may be fluorescent labels or other labels which are visualisable in tissues and which may be used for cell sorting.

In a preferred embodiment, a protein assay may be used for measuring intracellular concentrations of the cleaved Notch domain, concentrations of Notch extracellular domain; or concentrations of cleaved Delta extracellular domain in body fluids such as serum or from tissues from biopsies. In an alternative embodiment, a protein assay will be used for measuring expression of Notch signalling target genes (as described in more detail below).

Nucleic Acid Assays

Activation of the Notch receptor catalyses a series of downstream reactions leading to changes in the levels of expression of certain well defined genes. Thus, modulated Notch signalling can be assessed at the nucleic acid level by measuring, for example, intracellular concentrations of specific mRNAs. The advantage of using a nucleic acid assay is that such assays are sensitive and that small samples can be analysed.

The intracellular concentration of a particular mRNA, measured at any given time, reflects the level of expression of the corresponding gene at that time. Thus, levels of mRNA of downstream target genes of the Notch signalling pathway can be measured as an indirect assay for T-cell reactivity.

Various nucleic acid assays are known. Any conventional technique which is known or which is subsequently disclosed may be employed. Examples of suitable nucleic acid assay are mentioned below and include amplification, PCR, RT-PCR, RNase protection, blotting, spectrometry, reporter gene assays, gene chip arrays and other hybridization methods.

In particular, gene presence, amplification and/or expression may be measured in a sample directly, for example, by conventional Southern blotting, Northern blotting to quantitate the transcription of mRNA, dot blotting (DNA or RNA analysis), or in situ hybridisation, using an appropriately labelled probe. Those skilled in the art will readily envisage how these methods may be modified, if desired.

PCR was originally developed as a means of amplifying DNA from an impure sample. The technique is based on a temperature cycle which repeatedly heats and cools the reaction solution allowing primers to anneal to target sequences and extension of those primers for the formation of duplicate daughter strands. RT-PCR uses an RNA template for generation of a first strand cDNA with a reverse transcriptase. The cDNA is then amplified according to standard PCR protocol. Repeated cycles of synthesis and denaturation result in an exponential increase in the number of copies of the target DNA produced. However, as reaction components become limiting, the rate of amplification decreases until a plateau is reached and there is little or no net increase in PCR product. The higher the starting copy number of the nucleic acid target, the sooner this “end-point” is reached. Primers can be designed using standard procedures in the art, for example the Taqman technique.

Real-time PCR uses probes labeled with a fluorescent tag and differs from end-point PCR for quantitative assays in that it is used to detect PCR products as they accumulate rather than for the measurement of product accumulation after a fixed number of cycles. The reactions are characterized by the point in time during cycling when amplification of a target sequence is first detected through a significant increase in fluorescence. An advantage of real-time PCR is its accuracy in determining the amounts if target sequences in a sample. Suitable protocols are described, for example, in Meuer S. et al (2000).

The ribonuclease protection (RNase protection) assay is an extremely sensitive technique for the quantitation of specific RNAs in solution. The ribonuclease protection assay can be performed on total cellular RNA or poly(A)-selected mRNA as a target. The sensitivity of the ribonuclease protection assay derives from the use of a complementary in vitro transcript probe which is radiolabeled to high specific activity. The probe and target RNA are hybridized in solution, after which the mixture is diluted and treated with ribonuclease (RNase) to degrade all remaining single-stranded RNA. The hybridized portion of the probe will be protected from digestion and can be visualized via electrophoresis of the mixture on a denaturing polyacrylamide gel followed by autoradiography. Since the protected fragments are analyzed by high resolution polyacrylamide gel electrophoresis, the ribonuclease protection assay can be employed to accurately map mRNA features. If the probe is hybridized at a molar excess with respect to the target RNA, then the resulting signal will be directly proportional to the amount of complementary RNA in the sample.

PCR technology as described e.g. in section 14 of Sambrook et al., 1989, requires the use of oligonucleotide probes that will hybridise to nucleic acid. Strategies for selection of oligonucleotides are described below.

As used herein, a probe is e.g. a single-stranded DNA or RNA that has a sequence of nucleotides that includes between 10 and 50, preferably between 15 and 30 and most preferably at least about 20 contiguous bases that are the same as (or the complement of) an equivalent or greater number of contiguous bases. The nucleic acid sequences selected as probes should be of sufficient length and sufficiently unambiguous so that false positive results are minimised. The nucleotide sequences are usually based on conserved or highly homologous nucleotide sequences or regions of polypeptides. The nucleic acids used as probes may be degenerate at one or more positions.

Preferred regions from which to construct probes include 5′ and/or 3′ coding sequences, sequences predicted to encode ligand binding sites, and the like. For example, either the full-length cDNA clone disclosed herein or fragments thereof can be used as probes. Preferably, nucleic acid probes of the invention are labelled with suitable label means for ready detection upon hybridisation. For example, a suitable label means is a radiolabel. The preferred method of labelling a DNA fragment is by incorporating α³²P dATP with the Klenow fragment of DNA polymerase in a random priming reaction, as is well known in the art. Oligonucleotides are usually end-labelled with γ³²P-labelled ATP and polynucleotide kinase. However, other methods (e.g. non-radioactive) may also be used to label the fragment or oligonucleotide, including e.g. enzyme labelling, fluorescent labelling with suitable fluorophores and biotinylation.

Preferred are such sequences, probes which hybridise under high-stringency conditions. Stringency of hybridisation refers to conditions under which polynucleic acids hybrids are stable. Such conditions are evident to those of ordinary skill in the field. As known to those of skill in the art, the stability of hybrids is reflected in the melting temperature (Tm) of the hybrid which decreases approximately 1 to 1.5° C. with every 1% decrease in sequence homology. In general, the stability of a hybrid is a function of sodium ion concentration and temperature. Typically, the hybridisation reaction is performed under conditions of higher stringency, followed by washes of varying stringency.

As used herein, high stringency refers to conditions that permit hybridisation of only those nucleic acid sequences that form stable hybrids in 1 M Na+ at 65-68° C. High stringency conditions can be provided, for example, by hybridisation in an aqueous solution containing 6×SSC, 5× Denhardt's, 1% SDS (sodium dodecyl sulphate), 0.1 Na+ pyrophosphate and 0.1 mg/ml denatured salmon sperm DNA as non specific competitor. Following hybridisation, high stringency washing may be done in several steps, with a final wash (about 30 min) at the hybridisation temperature in 0.2-0.1×SSC, 0.1% SDS.

It is understood that these conditions may be adapted and duplicated using a variety of buffers, e.g. formamide-based buffers, and temperatures. Denhardt's solution and SSC are well known to those of skill in the art as are other suitable hybridisation buffers (see, e.g. Sambrook, et al., eds. (1989) Molecular Cloning: A Laboratory Manual, Cold Spring Harbor Laboratory Press, New York or Ausubel, et al., eds. (1990) Current Protocols in Molecular Biology, John Wiley & Sons, Inc.). Optimal hybridisation conditions have to be determined empirically, as the length and the GC content of the hybridising pair also play a role.

Gene expression may also be detected using a reporter system. Such a reporter system may comprise a readily identifiable marker under the control of an expression system, e.g. of the gene being monitored. Fluorescent markers, which can be detected and sorted by FACS, are preferred. Especially preferred are GFP and luciferase.

In general, reporter constructs useful for detecting Notch signalling by expression of a reporter gene may be-constructed the general teaching of Sambrook et al (1989). Typically, constructs according to the invention comprise a promoter by the gene of interest, and a coding sequence encoding the desired reporter constructs, for example of GFP or luciferase. Vectors encoding GFP and luciferase are known in the art and available commercially.

Sorting of cells, based upon detection of expression of genes, may be performed by any technique known in the art, as exemplified above. For example, cells may be sorted by flow cytometry or FACS. For a general reference, see Flow Cytometry and Cell Sorting: A Laboratory Manual (1992) A. Radbruch (Ed.), Springer Laboratory, New York.

Flow cytometry is a powerful method for studying and purifying cells. It has found wide application, particularly in immunology and cell biology: however, the capabilities of the FACS can be applied in many other fields of biology. The acronym F.A.C.S. stands for Fluorescence Activated Cell Sorting, and is used interchangeably with “flow cytometry”. The principle of FACS is that individual cells, held in a thin stream of fluid, are passed through one or more laser beams, causing light to be scattered and fluorescent dyes to emit light at various frequencies. Photomultiplier tubes (PMT) convert light to electrical signals, which are interpreted by software to generate data about the cells. Sub-populations of cells with defined characteristics can be identified and automatically sorted from the suspension at very high purity (˜100%).

FACS machines collect fluorescence signals in one to several channels corresponding to different laser excitation and fluorescence emission wavelengths. Fluorescent labelling allows the investigation of many aspects of cell structure and function. The most widely used application is immunofluorescence: the staining of cells with antibodies conjugated to fluorescent dyes such as fluorescein and phycoerythrin. This method is often used to label molecules on the cell surface, but antibodies can also be directed at targets within the cell. In direct immunofluorescence, an antibody to a particular molecule is directly conjugated to a fluorescent dye. Cells can then be stained in one step. In indirect immunofluorescence, the primary antibody is not labelled, but a second fluorescently conjugated antibody is added which is specific for the first antibody: for example, if the anti-polypeptide antibody is a mouse IgG, then the second antibody could be a rat or rabbit antibody raised against mouse IgG.

FACS can be used to measure gene expression in cells transfected with recombinant DNA encoding polypeptides. This can be achieved directly, by labelling of the protein product, or indirectly by using a reporter gene in the construct. Examples of reporter genes are β-galactosidase and Green Fluorescent Protein (GFP). β-galactosidase activity can be detected by FACS using fluorogenic substrates such as fluorescein digalactoside (FDG). FDG is introduced into cells by hypotonic shock, and is cleaved by the enzyme to generate a fluorescent product, which is trapped within the cell. One enzyme can therefore generate a large amount of fluorescent product. Cells expressing GFP constructs will fluoresce without the addition of a substrate. Mutants of GFP are available which have different excitation frequencies, but which emit fluorescence in the same channel. In a two-laser FACS machine, it is possible to distinguish cells which are excited by the different lasers and therefore assay two transfections at the same time.

Alternative means of cell sorting may also be employed. For example, the invention comprises the use of nucleic acid probes complementary to mRNA. Such probes can be used to identify cells expressing polypeptides individually, such that they may subsequently be sorted either manually, or using FACS sorting. Nucleic acid probes complementary to mRNA may be prepared according to the teaching set forth above, using the general procedures as described by Sambrook et al (1989).

In a preferred embodiment, the invention comprises the use of an antisense nucleic acid molecule, complementary to a mRNA, conjugated to a fluorophore which may be used in FACS cell sorting.

Methods have also been described for obtaining information about gene expression and identity using so-called gene chip arrays or high density DNA arrays (Chee). These high density arrays are particularly useful for diagnostic and prognostic purposes. Use may also be made of In Vivo Expression Technology (IVET) (Camilli). IVET identifies genes up-regulated during say treatment or disease when compared to laboratory culture.

Target Genes

In a preferred embodiment of the present invention, Notch signalling modulation and/or modulation of T-cell reactivity may be monitored by detecting down-stream modulation of expression of one or more target genes. The target genes of use in the nucleic acid or protein assays of the present invention may be endogenous target genes (i.e. endogenous target genes of the Notch signalling pathway) or synthetic reporter genes.

Endogenous Target Genes

Endogenous target genes of the Notch signalling pathway identified to date include, but are not limited to, Deltex, Suppressor of Deltex, genes of the Hes family (Hes-1 in particular), Enhancer of Split [E(spl)] complex genes, cytokines such as IL-10, CD antigens such as CD-23 and CD-4, CTLA-4, Dll-1, Numb, members of the Fringe group, Mastermind, Dsh and Dlx-1, homologues, variants and derivatives thereof. Although all genes the expression of which is modulated by Notch signalling may be used for the purpose of the present invention, preferred endogenous target genes are described below.

Deltex, an intracellular docking protein, replaces Su(H) as it leaves its site of interaction with the intracellular tail of Notch, as shown in FIG. 6. Deltex is a cytoplasmic protein containing a zinc-finger (Artavanis-Tsakonas; Osborne). It interacts with the ankyrin repeats of the Notch intracellular domain. Studies indicate that Deltex promotes Notch pathway activation by interacting with Grb2 and modulating the Ras-JNK signalling pathway (Matsuno). Deltex also acts as a docking protein which prevents Su(H) from binding to the intracellular tail of Notch (Matsuno). Thus, Su(H) is released into the nucleus where it acts as a transcriptional modulator. Recent evidence also suggests that, in a vertebrate B-cell system, Deltex, rather than the Su(H) homologue CBF1, is responsible for inhibiting E47 function (Ordentlich). Expression of Deltex is upregulated as a result of Notch activation in a positive feedback loop. The sequence of Homo sapiens Deltex (DTX1) mRNA may be found in GenBank Accession No. AF053700.

Hes-1 (Hairy-enhancer of Split-1) (Takebayahi) is a transcriptional factor with a basic helix-loop-helix structure. It binds to an important functional site in the CD4 silencer leading to repression of CD4 gene expression. Thus, Hes-1 is strongly involved in the determination of T-cell fate. Other genes from the Hes family include Hes-5 (mammalian Enhancer of Split homologue), the expression of which is also upregulated by Notch activation, and Hes-3. Expression of Hes-1 is upregulated as a result of Notch activation. The sequence of Mus musculus Hes-1 can be found in GenBank Accession No. D16464. The sequence of human Hes-1 can be found in GenBank Accession Nos. AK000415 and AF264785.

The E(spl) gene complex [E(spl)-C] (Leimeister) comprises seven genes of which only E(spl) and Groucho show visible phenotypes when mutant. E(spl) was named after its ability to enhance Split mutations, Split being another name for Notch. Indeed, E(spl)-C genes repress Delta through regulation of achaete-scute complex gene expression. Expression of E(spl) is upregulated as a result of Notch activation.

IL-10 (interleukin-10) is a factor produced by Th2 helper T-cells. It is a co-regulator of mast cell growth and shows extensive homology with the Epstein-Barr bcrfi gene. Although it is not clear whether or not IL-10 is a direct downstream target of the Notch signalling pathway, its expression has been found to be strongly upregulated coincident with Notch activation and is associated with immune tolerance. The IL-10 promoter region also has consensus CBF-1 binding sites. The mRNA sequence of IL-10 may be found in GenBank ref. No. G11041812.

CD-23 is the human leukocyte differentiation antigen CD23 (FCE2) which is a key molecule for B-cell activation and growth. It is the low-affinity receptor for IgE. Furthermore, the truncated molecule can be secreted, then functioning as a potent mitogenic growth factor. Although it is not thought to be a direct downstream target of the Notch signalling pathway, its expression has been found to be strongly upregulated coincident with Notch activation. The sequence for CD-23 may be found in GenBank ref. No. GI1783344.

Dlx-1 (distalless-1) (McGuiness) expression is downregulated as a result of Notch activation. Sequences for Dlx genes may be found in GenBank Accession Nos. U51000-3.

CTLA4 (cytotoxic T-lymphocyte activated protein 4) is an accessory molecule found on the surface of T-cells which is thought to play a role in the regulation of airway inflammatory cell recruitment and T-helper cell differentiation after allergen inhalation. The promoter region of the gene encoding CTLA4 has CBF1 response elements and its expression is upregulated as a result of Notch activation. The sequence of CTLA4 can be found in GenBank Accession No. L15006.

CD-4 expression is downregulated as a result of Notch activation. A sequence for the CD-4 antigen may be found in GenBank Accession No. XM006966.

Alternative target genes include Notch ligands such as Delta 1-4, Serrate 1 and 2 and Scabrous and homologues, variants and derivatives thereof, Notch receptors including Notch 1-4 and homologues, variants and derivatives thereof and transducers of the Notch signalling pathway such as CBF-1, Su(H) and members of the Ras-Jnk pathway. It is indeed thought that a positive feedback loop exists within the Notch signalling pathway whereby activation of the Notch receptor by binding of a Notch ligand will eventually lead to upregulated Notch and Notch ligand expression.

Synthetic Reporter Genes

In an alternative embodiment of the present invention, a synthetic reporter gene may be used for detecting Notch signalling modulation and/or T-cell reactivity. The reporter gene will preferably be under the transcriptional control of a promoter region or responder element(s) sensitive to Notch signalling.

A wide variety of reporters may be used in the assay methods (as well as screens) of the present invention with preferred reporters providing conveniently detectable signals (eg. by spectroscopy). By way of example, a reporter gene may encode an enzyme which catalyses a reaction which alters light absorption properties.

Other protocols include enzyme-linked immunosorbent assay (ELISA), radioimmunoassay (RIA) and fluorescent activated cell sorting (FACS). A two-site, monoclonal-based immunoassay utilising monoclonal antibodies reactive to two non-interfering epitopes may even be used. These and other assays are described, among other places, in Hampton R et al (1990, Serological Methods, A Laboratory Manual, APS Press, St Paul Minn.) and Maddox D E et al.

One skilled in the art will recognize that the identity of the specific reporter gene can, of course, vary. Examples of reporter genes that have been used in the art include, but are not limited to, genes encoding an enzymatic activity such as chloramphenicol acetyltransferase (CAT) gene, Green Fluorescent Protein (GFP), luciferase (luc), β-galactosidase, invertase, horseradish peroxidase, glucuronidase, exo-glucanase, glucoamylase or alkaline phosphatase. Alternatively, the reporter gene may comprise a radiolabel or a fluorescent label such as FITC, rhodamine, lanthanide phosphors, or a green fluorescent fusion protein (See for example Stauber et al). Alternatively, the reporter may comprise a predetermined polypeptide epitope which can be recognized by a secondary reporter such as leucine zipper pair sequences, binding sites for secondary antibodies, metal binding domains, or epitope tags. One skilled in the art will appreciate that the specific reporter gene or genes utilized in the methods disclosed herein may vary and may also depend on the specific model system utilized, and the methods disclosed herein are not limited to any specific reporter gene or genes.

By way of further examples, a number of companies such as Pharmacia Biotech (Piscataway, N.J.), Promega (Madison, Wis.), and US Biochemical Corp (Cleveland, Ohio) supply commercial kits and protocols for assay procedures. Suitable reporter molecules or labels include those radionuclides, enzymes, fluorescent, chemiluminescent, or chromogenic agents as well as substrates, cofactors, inhibitors, magnetic particles and the like. Patents teaching the use of such labels include U.S. Pat. No. 3,817,837; U.S. Pat. No. 3,850,752; U.S. Pat. No. 3,939,350; U.S. Pat. No. 3,996,345; U.S. Pat. No. 4,277,437; U.S. Pat. No. 4,275,149 and U.S. Pat. No. 4,366,241.

The reporter gene used in the method of the present invention is under the transcriptional control of at least one Notch signalling sensitive promoter region and/or responder element. Promoter regions and/or responder elements sensitive to Notch signalling include the regulatory elements of endogenous Notch target genes such as the HES promoters, Deltex promoter, Notch and Notch ligand promoters, IL-10 promoters. Regulatory elements of use in the present invention also include single or multimerized CBF1 sites and CTLA4 promoters. The regulatory elements are positioned such that activation of the Notch signalling pathway results in increased expression of the reporter gene.

One or more copies of the reporter gene can be inserted into the host cell by methods known in the art. The term “host cell”—in relation to the present invention includes any cell that could comprise the target for the agent of the present invention. Polynucleotides may be introduced into prokaryotic cells or eukaryotic cells, for example yeast, insect or mammalian cells.

Polynucleotides of the invention may be introduced into suitable host cells using a variety of techniques known in the art, such as transfection, transformation and electroporation. Where polynucleotides of the invention are to be administered to animals, several techniques are known in the art, for example infection with recombinant viral vectors such as retroviruses, herpes simplex viruses and adenoviruses, direct injection of nucleic acids and biolistic transformation.

In the present invention, the host cells will preferably be mammalian cells and the polypeptides will be expressed either intracellularly, on the cell membranes or secreted in a culture media if preceded by an appropriate leader sequence. Preferably, the host cell will be a cell of the immune system.

Cells of the Immune System

Cells of use in the present invention are cells of the immune system capable of transducing the Notch signalling pathway.

Most preferably the cells of use in the present invention are T-cells. These include, but are not limited to, CD4⁺ and CD8⁺ mature T-cells, immature T-cells of peripheral or thymic origin and NK-T-cells.

Alternatively, the cells will be antigen-presenting cells (APCs). APCs include dendritic cells (DCs) such as interdigitating DCs or follicular DCs, Langerhans cells, PBMCs, macrophages, B-lymphocytes, T-lymphocytes, or other cell types such as epithelial cells, fibroblasts or endothelial cells, constitutively expressing or activated to express a MHC Class II molecules on their surfaces. Precursors of APCs include CD34+ cells, monocytes, fibroblasts and endothelial cells. The APCs or precursors may be modified by the culture conditions or may be genetically modified, for instance by transfection of one or more genes.

The T-cells or APCs may be isolated from a patient, or from a donor individual or another individual. The cells are preferably mammalian cells such as human or mouse cells. Preferably the cells are of human origin. The APC or precursor APC may be provided by a cell proliferating in culture such as an established cell line or a primary cell culture. Examples include hybridoma cell lines, L-cells and human fibroblasts such as MRC-5. Preferred cell lines for use in the present invention include Jurkat, H9, CEM and EL4 T-cells; long-term T-cell clones such as human HA1.7 or mouse D10 cells; T-cell hybridomas such as DO11.10 cells; macrophage-like cells such as U937 or THP1 cells; B-cell lines such as EBV-transformed cells such as Raji, A20 and M1 cells.

Dendritic cells (DCs) can be isolated/prepared by a number of means, for example they can either be purified directly from peripheral blood, or generated from CD34⁺ precursor cells for example after mobilisation into peripheral blood by treatment with GM-CSF, or directly from bone marrow. From peripheral blood, adherent precursors can be treated with a GM-CSF/IL-4 mixture (Inaba et al), or from bone marrow, non-adherent CD34⁺ cells can be treated with GM-CSF and TNF-3 (Caux et al). DCs can also be routinely prepared from the peripheral blood of human volunteers, similarly to the method of Sallusto and Lanzavecchia (1994) using purified peripheral blood mononucleocytes (PBMCs) and treating 2 hour adherent cells with GM-CSF and IL-4. If required, these may be depleted of CD19⁺ B-cells and CD3⁺, CD2⁺ T-cells using magnetic beads (Coffin et al). Culture conditions may include other cytokines such as GM-CSF or IL-4 for the maintenance and, or activity of the dendritic cells or other antigen presenting cells.

T-cells and B-cells of use in the invention are preferably obtained from cell lines such as lymphoma or leukemia cell lines, T-cell hybridomas or B-cell hybridomas but may also be isolated from an individual suffering from a disease of the immune system or a recipient for a transplant operation or from a related or unrelated donor individual. T-cells and B-cells may be obtained from blood or another source (such as lymph nodes, spleen, or bone marrow) and may be enriched or purified by standard procedures. Alternatively whole blood may be used or leukocyte enriched blood or purified white blood cells as a source of T-cells and other cell types. It is particularly preferred to use helper T-cells (CD4⁺). Alternatively other T-cells such as CD8⁺ cells may be used.

For assays involving monitoring or detection of the reactivity of a T-cell or T-cell population to an antigen for use in clinical applications, the assay will generally involve removal of a sample (body fluids such as plasma, serum or tissue extracts from biopsies) from a patient prior to the step of detecting a signal resulting from modulation of Notch signalling. When monitoring or detecting the reactivity of a T-cell or T-cell population to an antigen, the T-cell or T-cell population will preferably be isolated from an individual that has undergone therapy aimed at modulating the reactivity of said T-cells to a given antigen (self or non-self).

Preferably the detected signal is compared with a reference signal and any modulation with respect to the reference signal measured. Thus, a diagnostic assay in accordance with the invention may detect and monitor Notch signalling compared to normal control samples to detect the presence of tolerised or highly reactive T-cells.

Arrays

It will be appreciated that more than one indicator, signal or marker from the Notch signalling pathway may be detected by use of the assay. For example, two, three, four, five, ten, twelve, fifteen or more indicators may be measured or detected simultaneously, separately or sequentially to give a profile or “fingerprint” of the degree of activation of the Notch signalling pathway. Such a profile gives a sophisticated measure of the state of the immune system at a given time. Such profiles may conveniently be generated, for example, by use of protein or nucleic acid chip.

Thus in a further aspect of the invention, there is provided a nucleic acid array consisting essentially of a least two, suitably at least three, suitably at least five, for example at least ten, twelve or fifteen sequences specific for nucleic acid indicators of the Notch signalling pathway. The sequences used may be conventional nucleic acid sequences or may be, for example, derivatives thereof having increased stability.

In a further aspect of the invention, there is provided a protein or polypeptide binding array consisting essentially of a least two, suitably at least three, suitably at least five, for example at least ten, twelve or fifteen protein or polypeptide binding agents or sequences specific for protein or polypeptide indicators of the Notch signalling pathway. It will be appreciated that mixed or hybrid arrays with combinations of nucleic acid and protein binding agents are also within the scope of the invention.

The term “consisting essentially of” as used herein means that the array bears sequences of the type described alone substantially without unrelated binding agents or sequences. Such pre-selected arrays have the advantages that they may be cheaper to make and/or use than arrays which also display unrelated sequences or binding agents. However, it will be appreciated that arrays which display unrelated binding agents or sequences may also be used in the invention if preferred.

Candidate Modulator Screens

The invention additionally provides a method of screening for a candidate modulator of Notch signalling, the method comprising mixing in a buffer an appropriate amount of functional Notch, and a candidate modulator; and monitoring for any modulation of Notch signalling.

The term “modulate” as used herein refers to a change or alteration in the biological activity of the Notch signalling pathway or a target signalling pathway thereof. The term “modulator” may refer to antagonists or inhibitors of Notch signalling, i.e. compounds which block, at least to some extent, the normal biological activity of the Notch signalling pathway. Conveniently such compounds may be referred to herein as inhibitors or antagonists. Alternatively, the term “modulator” may refer to agonists of Notch signalling, i.e. compounds which stimulate or upregulate, at least to some extent, the normal biological activity of the Notch signalling pathway. Conveniently such compounds may be referred to as upregulators or agonists.

The candidate modulator of the present invention may be an organic compound or other chemical. In this embodiment, the candidate modulator will be an organic compound comprising two or more hydrocarbyl groups. Here, the term “hydrocarbyl group” means a group comprising at least C and H and may optionally comprise one or more other suitable substituents. Examples of such substituents may include halo-, alkoxy-, nitro-, an alkyl group, a cyclic group etc. In addition to the possibility of the substituents being a cyclic group, a combination of substituents may form a cyclic group. If the hydrocarbyl group comprises more than one C then those carbons need not necessarily be linked to each other. For example, at least two of the carbons may be linked via a suitable element or group. Thus, the hydrocarbyl group may contain hetero atoms. Suitable hetero atoms will be apparent to those skilled in the art and include, for instance, sulphur, nitrogen and oxygen. The candidate modulator may comprise at least one cyclic group. The cyclic group may be a polycyclic group, such as a non-fused polycyclic group. For some applications, the agent comprises at least the one of said cyclic groups linked to another hydrocarbyl group.

In a preferred embodiment, the candidate compound will be an amino acid sequence or a chemical derivative thereof, or a combination thereof. In another preferred embodiment, the candidate compound will be a nucleotide sequence—which may be a sense sequence or an anti-sense sequence. The candidate modulator may also be an antibody.

Candidate modulators may be synthetic compounds or natural isolated compounds.

Assays for identifying novel modulators of Notch signalling comprise the steps of a) providing a candidate modulator; b) providing a Notch receptor or a functional variant thereof; c) bringing Notch and the candidate modulator together under condidtions such that the modulator interacts with Notch; and d) detecting modulation of Notch signalling using any of the assay methods described above.

Preferably, step d) will comprise a screen, whereby a number of agents are tested. In one aspect, the assay method of the present invention is a high throughput screen (HTS).

Techniques for drug screening may be based on the method described in Geysen, European Patent No. 0138855, published on Sep. 13, 1984. In summary, large numbers of different small peptide candidate modulators are synthesized on a solid substrate, such as plastic pins or some other surface. The peptide test compounds are reacted with a suitable target or fragment thereof and washed. Bound entities are then detected—such as by appropriately adapting methods well known in the art. A purified target can also be coated directly onto plates for use in drug screening techniques. Plates of use for high throughput screening (HTS) will be multi-well plates, preferably having 96, 384 or over 384 wells/plate. Cells can also be spread as “lawns”. Alternatively, non-neutralising antibodies can be used to capture the peptide and immobilise it on a solid support. High throughput screening, as described above for synthetic compounds, can also be used for identifying organic candidate modulators.

This invention also contemplates the use of competitive drug screening assays in which neutralising antibodies capable of binding a target specifically compete with a test compound for binding to a target.

It is expected that the assay methods of the present invention will be suitable for both small and large-scale screening of test compounds as well as in quantitative assays.

Polypeptide Sequences

As used herein, the term “polypeptide” is synonymous with the term “amino acid sequence” and/or the term “protein”. In some instances, the term “polypeptide” is synonymous with the term “peptide”.

“Peptide” usually refers to a short amino acid sequence that is 10 to 40 amino acids long, preferably 10 to 35 amino acids.

The polypeptide sequence may be prepared and isolated from a suitable source, or it may be made synthetically or it may be prepared by use of recombinant DNA techniques.

Polynucleotide Sequences

As used herein, the term “polynucleotide sequence” is synonymous with the term “polynucleotide” and/or the term “nucleotide sequence”.

The polynucleotide sequence may be DNA or RNA of genomic or synthetic or of recombinant origin. They may also be cloned by standard techniques. The polynucleotide sequence may be double-stranded or single-stranded whether representing the sense or antisense strand or combinations thereof.

“Polynucleotide” refers to a polymeric form of nucleotides of at least 10 bases in length and up to 1,000 bases or even more. Longer polynucleotide sequences will generally be produced using recombinant means, for example using a PCR (polymerase chain reaction) cloning techniques. This will involve making a pair of primers (e.g. of about 15 to 30 nucleotides) flanking a region of the targeting sequence which it is desired to clone, bringing the primers into contact with mRNA or cDNA obtained from an animal or human cell, performing a polymerase chain reaction (PCR) under conditions which bring about amplification of the desired region, isolating the amplified fragment (e.g. by purifying the reaction mixture on an agarose gel) and recovering the amplified DNA. The primers may be designed to contain suitable restriction enzyme recognition sites so that the amplified DNA can be cloned into a suitable cloning vector.

The nucleic acid may be RNA or DNA and is preferably DNA. Where it is RNA, manipulations may be performed via cDNA intermediates. Generally, a nucleic acid sequence encoding the first region will be prepared and suitable restriction sites provided at the 5′ and/or 3′ ends. Conveniently the sequence is manipulated in a standard laboratory vector, such as a plasmid vector based on pBR322 or pUC19 (see below). Reference may be made to Molecular Cloning by Sambrook et al. (Cold Spring Harbor, 1989) or similar standard reference books for exact details of the appropriate techniques.

Sources of nucleic acid may be ascertained by reference to published literature or databanks such as GenBank. Nucleic acid encoding the desired first or second sequences may be obtained from academic or commercial sources where such sources are willing to provide the material or by synthesising or cloning the appropriate sequence where only the sequence data are available. Generally this may be done by reference to literature sources which describe the cloning of the gene in question.

Alternatively, where limited sequence data is available or where it is desired to express a nucleic acid homologous or otherwise related to a known nucleic acid, exemplary nucleic acids can be characterised as those nucleotide sequences which hybridise to the nucleic acid sequences known in the art.

The polynucleotide sequence may comprise, for example, a protein-encoding domain, an antisense sequence or a functional motif such as a protein-binding domain and includes variants, derivatives, analogues and fragments thereof. The term also refers to polypeptides encoded by the nucleotide sequence.

Variants, Derivatives, Analogues, Homologues and Fragments

In addition to the specific polypeptide and polynucleotide sequences mentioned herein, the present invention also encompasses the use of variants, derivatives, analogues, homologues, mimetics and fragments thereof.

In the context of the present invention, a variant of any given sequence is a sequence in which the specific sequence of residues (whether amino acid or nucleic acid residues) has been modified in such a manner that the polypeptide or polynucleotide in question retains at least one of its endogenous functions. A variant sequence can be modified by addition, deletion, substitution modification replacement and/or variation of at least one residue present in the naturally-occurring protein.

The term “derivative” as used herein, in relation to proteins or polypeptides of the present invention includes any substitution of, variation of, modification of, replacement of, deletion of and/or addition of one (or more) amino acid residues from or to the sequence providing that the resultant protein or polypeptide retains at least one of its endogenous functions.

The term “analogue” as used herein, in relation to polypeptides or polynucleotides, includes any polypeptide or polynucleotide which retains at least one of the functions of the endogenous polypeptide or polynucleotide but generally has a different evolutionary origin thereto.

The term “mimetic” as used herein, in relation to polypeptides or polynucleotides, refers to a chemical compound that possesses at least one of the endogenous functions of the polypeptide or polynucleotide which it mimics.

Typically, amino acid substitutions may be made, for example from 1, 2 or 3 to 10 or 20 substitutions provided that the modified sequence retains the required transport activity or ability to modulate Notch signalling. Amino acid substitutions may include the use of non-naturally occurring analogues.

Proteins of use in the present invention may also have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity, and/or the amphipathic nature of the residues as long as the transport or modulation function is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar-uncharged | C S T M |
| | | N Q |
| | Polar-charged | D E |
| | | K R |
| AROMATIC | | H F W Y |

As used herein, the term “protein” includes single-chain polypeptide molecules as well as multiple-polypeptide complexes where individual constituent polypeptides are linked by covalent or non-covalent means. As used herein, the terms “polypeptide” and “peptide” refer to a polymer in which the monomers are amino acids and are joined together through peptide or disulfide bonds. The terms subunit and domain may also refer to polypeptides and peptides having biological function.

“Fragments” are also variants and the term typically refers to a selected region of the polypeptide or polynucleotide that is of interest either functionally or, for example, in an assay. “Fragment” thus refers to an amino acid or nucleic acid sequence that is a portion of a full-length polypeptide or polynucleodtide.

Such variants may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site. The flanking regions will contain convenient restriction sites corresponding to sites in the naturally-occurring sequence so that the sequence may be cut with the appropriate enzyme(s) and the synthetic DNA ligated into the cut. The DNA is then expressed in accordance with the invention to make the encoded protein. These methods are only illustrative of the numerous standard techniques known in the art for manipulation of DNA sequences and other known techniques may also be used.

Polynucleotide variants will preferably comprise codon optimised sequences. Codon optimisation is known in the art as a method of enhancing RNA stability and therefor gene expression. The redundancy of the genetic code means that several different codons may encode the same amino-acid. For example, Leucine, Arginine and Serine are each encoded by six different codons. Different organisms show preferences in their use of the different codons. Viruses such as HIV, for instance, use a large number of rare codons. By changing a nucleotide sequence such that rare codons are replaced by the corresponding commonly used mammalian codons, increased expression of the sequences in mammalian target cells can be achieved. Codon usage tables are known in the art for mammalian cells, as well as for a variety of other organisms. Preferably, at least part of the sequence is codon optimised. Even more preferably, the sequence is codon optimised in its entirety.

As used herein, the term “homology” can be equated with “identity”. An homologous sequence will be taken to include an amino acid sequence which may be at least 75, 85 or 90% identical, preferably at least 95 or 98% identical. In particular, homology should typically be considered with respect to those regions of the sequence (such as amino acids at positions 51, 56 and 57) known to be essential for an activity. Although homology can also be considered in terms of similarity (i.e. amino acid residues having similar chemical properties/functions), in the context of the present invention it is preferred to express homology in terms of sequence identity.

Homology comparisons can be conducted by eye, or more usually, with the aid of readily available sequence comparison programs. These commercially available computer programs can calculate % homology between two or more sequences.

Percent homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package (see below) the default gap penalty for amino acid sequences is —12 for a gap and —4 for each extension.

Calculation of maximum % homology therefor firstly requires the production of an optimal alignment, taking into consideration gap penalties. A suitable computer program for carrying out such an alignment is the GCG Wisconsin Bestfit package (Devereux). Examples of other software than can perform sequence comparisons include, but are not limited to, the BLAST package, FASTA (Atschul) and the GENEWORKS suite of comparison tools. Both BLAST and FASTA are available for offline and online searching. However it is preferred to use the GCG Bestfit program.

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). It is preferred to use the public default values for the GCG package, or in the case of other software, the default matrix, such as BLOSUM62.

Once the software has produced an optimal alignment, it is possible to calculate % homology, preferably % sequence identity. The software typically does this as part of the sequence comparison and generates a numerical result.

Nucleotide sequences which are homologous to or variants of sequences of use in the present invention can be obtained in a number of ways, for example by probing DNA libraries made from a range of sources. In addition, other viral/bacterial, or cellular homologues particularly cellular homologues found in mammalian cells (e.g. rat, mouse, bovine and primate cells), may be obtained and such homologues and fragments thereof in general will be capable of selectively hybridising to the sequences shown in the sequence listing herein. Such sequences may be obtained by probing cDNA libraries made from or genomic DNA libraries from other animal species, and probing such libraries with probes comprising all or part of the reference nucleotide sequence under conditions of medium to high stringency. Similar considerations apply to obtaining species homologues and allelic variants of the amino acid and/or nucleotide sequences useful in the present invention.

Variants and strain/species homologues may also be obtained using degenerate PCR which will use primers designed to target sequences within the variants and homologues encoding conserved amino acid sequences within the sequences of use in the present invention. Conserved sequences can be predicted, for example, by aligning the amino acid sequences from several variants/homologues. Sequence alignments can be performed using computer software known in the art. For example the GCG Wisconsin PileUp program is widely used. The primers used in degenerate PCR will contain one or more degenerate positions and will be used at stringency conditions lower than those used for cloning sequences with single sequence primers against known sequences.

Alternatively, such nucleotide sequences may be obtained by site directed mutagenesis of characterised sequences. This may be useful where for example silent codon changes are required to sequences to optimise codon preferences for a particular host cell in which the nucleotide sequences are being expressed. Other sequence changes may be desired in order to introduce restriction enzyme recognition sites, or to alter the activity of the polynucleotide or encoded polypeptide.

In a first step of the method of the present invention, any one or more of the above candidate modulators is brought into contact with a cell of the immune system. Cells of the immune system of use in the present invention are described below.

The method of the present invention (in so far as it allows T-cell reactivity and immunotherapy to be monitored) and modulators identifiable by the method of the invention will be of use in therapy: for the treatment of diseases of the immune system and for monitoring such treatment.

Therapy

The term “therapy” includes curative effects, alleviation effects, and prophylactic effects. The therapy may be on humans or animals.

The present invention may be used in the treatment of disorders and/or conditions of the immune system including, in particular, T-cell mediated diseases or disorders. A detailed description of the conditions affected by the Notch signalling pathway may be found in our WO98/20142, WO00/36089 and WO/00135990.

Diseased or infectious states that may be described as being mediated by T-cells include, but are not limited to, any one or more of asthma, allergy, tumour induced aberrations to the T-cell system and infectious diseases such as those caused by Plasmodium species, Microfilariae, Helminths, Mycobacteria, HIV, Cytomegalovirus, Pseudomonas, Toxoplasma, Echinococcus, Haemophilus influenza type B, measles, Hepatitis C or Toxicara. Thus particular conditions that may be treated or prevented which are mediated by T-cells include multiple sclerosis, rheumatoid arthritis and diabetes. The present invention may also be used in organ transplantation. The present invention is also useful in treating immune disorders such as autoimmune disorders or graft rejection such as allograft rejection.

Pharmaceutical Compositions

The present invention provides a pharmaceutical composition comprising administering a therapeutically effective amount of at least one modulator identified by the method of the present invention and a pharmaceutically acceptable carrier, diluent or excipients (including combinations thereof).

The pharmaceutical compositions may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

There may be different composition/formulation requirements dependent on the different delivery systems. By way of example, the pharmaceutical composition of the present invention may be formulated to be delivered using a mini-pump or by a mucosal route, for example, as a nasal spray or aerosol for inhalation or ingestable solution, or parenterally in which the composition is formulated by an injectable form, for delivery, by, for example, an intravenous, intramuscular or subcutaneous route. Alternatively, the formulation may be designed to be delivered by both routes.

Where the compound is to be delivered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions can be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

Administration

Typically, a physician will determine the actual dosage which will be most suitable for an individual subject and it will vary with the age, weight and response of the particular patient. The dosages below are exemplary of the average case. There can, of course, be individual instances where higher or lower dosage ranges are merited.

The compositions of the present invention may be administered by direct injection. The composition may be formulated for parenteral, mucosal, intramuscular, intravenous, subcutaneous, intraocular or transdermal administration.

The composition of the present invention may be administered by viral or non-viral techniques. Viral delivery mechanisms include but are not limited to adenoviral vectors, adeno-associated viral (AAV) vectos, herpes viral vectors, retroviral vectors, lentiviral vectors, and baculoviral vectors. Non-viral delivery mechanisms include lipid mediated transfection, liposomes, immunoliposomes, lipofectin, cationic facial amphiphiles (CFAs) and combinations thereof. The routes for such delivery mechanisms include but are not limited to mucosal, nasal, oral, parenteral, gastrointestinal, topical, or sublingual routes.

The composition of the present invention may, in more detail, be administered, for example, as a nasal spray or aerosol for inhalation or as an ingestable solution; a parenteral route where delivery is by an injectable form, such as, for example, an intravenous, intramuscular, intradermal, intra-articular, intrathecal, intra-peritoneal or subcutaneous route, or via the alimentary tract (for example, via the Peyers patches).

The routes of administration and dosages described are intended only as a guide since a skilled practitioner will be able to determine readily the optimum route of administration and dosage for any particular patient depending on, for example, the age, weight and condition of the patient. Preferably the pharmaceutical compositions are in unit dosage form. The present invention includes both human and veterinary applications.

The present invention will now further be described by way of non-limiting examples.

### REFERENCE EXAMPLE 1

Induction of Tolerance—Delta Expressing Human T-Cells can Block the Response of Normal T-Cells.

As described in WO98/20142, an influenza-reactive human T-cell clone (HA1.7) was transfected with mouse Delta using a retroviral construct to allow cell surface expression of the Delta protein. Mixing of this cell population with normal HA1.7 prevented subsequent reactivity of these normal HA1.7 with peptide HA306-318 and antigen presenting cells. 5×10⁵ HA1.7 were mixed with 1×10⁶ irradiated DRB1*0101 peripheral blood mononuclear cells (PBMC)+1 microgram HA306-318 and cultured at 37EC. 6 hours later 5% lymphocult (IL-2 containing medium) was added in a total volume of 1 ml. 24 hours after the initiation of culture Delta or control retrovirus or nothing was added. 7 days after the start of culture, cells were harvested, washed and the transfected cells irradiated. The transfected cells were mixed at a ratio of 2:1 with untreated HA1.7 and cultured for 2 days. Mixed cultures were then harvested, washed and plated out using 2×10⁴ viable cells/well together with:

a) 2.5×10⁴DRB1*0101 PBMCs (medium),

b) 2.5×10⁴ DRB1*0101 PBMCs+peptide (Ag+APC) and

c) 5% lymphocult (IL-2).

Cells were harvested after 68 hours with the addition of tritiated thymidine for the final 8 hours. The results are shown in FIG. 1.

Following culture alone or with control virus transfected HA1.7, untreated HA1.7 responds well to peptide+antigen presenting cells. Incubation with Delta transfected irradiated HA1.7 completely prevents the response of untreated HA1.7 to antigen+APC. However, such cells respond as well as untreated or control virus incubated HA1.7 to IL-2, indicating that they are not simply unable to proliferate.

### REFERENCE EXAMPLE 2

Induction of Tolerance—Serrate Expression by Antigen Presenting Cells Prevents T-Cell Responses.

Clone HA1.7 was mixed with peptide HA306-318 (1.0 microgram/ml) in the presence of L cells expressing HLA-DRB1*0101 (as antigen presenting cells), using 2×10⁴ of each cell type. The L cells were transfected with either control (pure) or serrate (serrate L cells) expressing retrovirus. The proliferative response was measured after 72 hours following addition of tritiated thymidine for the last 8 hours of culture. Results are shown in FIG. 2 for HA1.7 cultures:

a) alone

b) +IL-2

c) +peptide+DRB1*0101-L cells

d) +peptide+DRB1*0101-L cells transfected with control virus+peptide+DRB1*0101-L cells transfected with serrate virus

HA1.7 stimulated by serrate expressing L cells responded poorly to antigen when compared with those stimulated by control transfected L cells.

### REFERENCE EXAMPLE 3

Induction of Tolerance—Serrate Expressing Antigen Presenting Cells Induce Regulatory T-Cells.

Clone HA1.7 was mixed with peptide HA306-318 and L cells (expressing DRB1*0101 as antigen presenting cells) in the presence of 2% IL-2. The L cells were transfected with either control or serrate expressing retrovirus. After 7 days in culture, the HA1.7 were harvested washed and irradiated before being mixed with fresh HA1.7 (using 2×10⁴ each population). Cells were cultured for a further 2 days before being stimulated with peptide (1.0 microgram/ml)+normal antigen presenting cells (DRB1*0101 PBMCs). The proliferative response was measured after 72 hours following addition of tritiated thymidine for the last 8 hours of culture. The results are shown in FIG. 3 for fresh HA1.7 cultured:

a) alone

b) +IL-2

c) +control virus induced HA1.7, then peptide+DRB1*0101 PBMC

d) +serrate virus induced HA1.7, then peptide+DRB1*0101 PBMC

HA1.7 induced by Serrate expressing L cells (Serrate induced HA1.7) prevented the response of normal HA1.7 to a normal antigenic stimulus. This shows the ability of cells tolerised by exposure to Serrate, to pass on their tolerance to a naive cell population (infectious/bystander tolerance).

### EXAMPLE 4

Detection of Tolerance—Notch Signalling Occurs During the Induction of Anergy. HA1.7—concentration of cells 2×10⁶/ml; activation conditions were anti-CD3 and anti-CD28 antibody. Anergy conditions were HA306-318 peptide at 25 μg/ml; concentration of peptide 25 μg/ml—was cultured for varying times as indicated in FIG. 4. Total RNA was produced by homogenisation of cells in guanidine isothiocynanate solution and separated over a CsCl gradient. The extracted RNA was subjected to real-time PCR using the following primers and probes:

HES-1 forward primer: CAT TCT GGA AAT GAC AGT GAA GCA

HES-1 reverse primer: CAG CGC AGC CGT CAT CT

HES-1 probe: CTC CGG AAC CTG CAG CGG GC

Deltex forward primer: TTC CCT CGC CAC TGC TAT CT

Deltex reverse primer: GAC TCG CCC GTG GTG TTG

Deltex probe: CCC AAC AAC GAG AAA GGC CGG AA

In FIG. 4 the expression of genes is shown relative to that of a control gene, GAPDH.

### EXAMPLE 5

Generation of CD4⁺ Regulatory T-Cells Following Peptide Induced Tolerance.

Mice were treated intranasally with saline or Der peptide p1, 110-131 to induce tolerance. Two weeks later mice CD4⁺ or CD8⁺ T-cells were purified from the spleens from both groups of mice. The cells were transferred to naive recipient mice at 1×10⁶, 5×10⁶ or 2×10⁷ cells per mouse. On the same day, recipients were immunised with 50 :g Der p 1/CFA. One week later draining lymph node cells were cultured in vitro with 10: g/ml Der p 1.

The results are illustrated in FIG. 9 which shows the T-cell proliferative of lymph node cells from mice receiving:

CD4⁺ T-cells from control mice (closed circle);

CD4⁺ from peptide tolerised animals (open square);

CD8⁺ T-cells from peptide tolerised animals (open triangle).

The results establish that intranasal administration of Der p1, 110-131 of Der p 1 leads to the induction of a CD4⁺ regulatory T-cell population which is consistent with the fact that this peptide contains the immunodominant epitope recognised by CD4⁺ T-cells in H-2^{b} mice.

### EXAMPLE 6

Phenotypic Changes of CD4⁺ T-Cells During the Induction of Tolerance.

Mice were immunised with Der p1, 110-131/CFA i.p. (i.e. activated) or with Der p1, 110-131 in saline intranasally (i.e. tolerised). Splenic CD4⁺ cells were purified by antibody purification on days 2, 4 and 8 post treatment. The cells were analyses by two colour flow cytometry using CD4⁺ together with either CD25 or CTLA-4.

In more detail, single cell suspension of lymph node of spleen was prepared in RPM1 1640 medium containing 2% FCS, followed by gentle desegregation through a 21-gauge needle. Cells were released from spleens and lymph nodes by mincing the tissues of sterile frosted glass glides and cell suspensions passed through a nylon sieve (0.8 μM). Red cells were removed by hypotonic lysis using sterile distilled water. Cells were counted by hemocytometer, suspended to appropriate concentrations in PBS+0.1% bovine serum albumin (PBS+0.1% BSA). Cells were stained in for 20 min on ice with fluorescein isothiocyanate-(FITC-), or phycoerythrin (PE-) conjugated antibodies in PBS+0.1% BSA. Two colour analysis was performed using a Becton Dickinson Facscalibur machine and data was analysed using cellquest software.

FIG. 10 shows representative FACS profiles obtained. These results demonstrate that CD4⁺ cells from mice undergoing tolerance induction revealed a rapid and sustained expression of CD25 over the 8 day period accompanied by a gradual increase in cell surface expression of CTLA-4.

### EXAMPLE 7

Transient Activation of CD4⁺ T-Cells Preceded the Development of Tolerance.

In a first instance (FIG. 11a), mice were treated intranasally with Der p1, 110-131 in saline. On days 2, 4, 8 and 14, spleen cells were removed and cultured in vitro with Der p1, 110-131. The supernatants were collected at 24 h and assayed for the presence of:

IL-2 (closed circles); and

IL-3 (open circles).

In more detail, cytokine measurements were made with the use of bioassays for IL-2 and IL-3. All cytokines were measured in culture supernatants taken at 24 h. The CTLL-2 cell line proliferates maximally with IL-2 but antigen-specific immune response and only poorly in the presence of IL-4. Test supernatants (50 ml volumes) were added to 5×10³ CTLL-2 cells (50 ml) per well and cultured for 24 h at 37° C. and pulsed for 6 h with [3 H]thymidine.

FD C-P1 cells proliferate maximally to IL-3 and granulocyte macrophage colony stimulating factor but poorly to IFN-γ. Cells (2×10³) were cultured with test supernatants for 24 h and pulsed with [3 H]thymidine.

In a second instance (FIG. 11b), CD4⁺ T-cells were purified from the spleen of mice receiving:

Der.p1, 110-131/CFA (i.e. activated—hatched bars); or

Der p1, 110-131 (i.e. tolerised—filled bars) in saline intranasally.

Cells were collected on days 2, 4 and 8 post treatment and total RNA was prepared and reverse transcribed to cDNA and then analysed by semi-quantative Real Time PCR (TaqMan) for the expression of IL-2, IL-3, IL-10 and TGF-31.

In more detail, 5 μg total RNA extracted from T-cells was incubated with 1.5 μg oligo(dT)₁₅-primer at 95° C. for 15 minutes then cooled down on ice for another 5 minutes. For first stand cDNA synthesis, 400U Superscript II reverse transcriptase (Gibco BRL) containing RT buffer, 10 mM DTT and dNTP mixture (50 μM dATP, dGTP, dTTP and 0.5 μM dCTP), were added and left at 42° C. for 90 minutes. Reaction was stopped and secondary structures denatured at 75° C. for 10 minutes. PCR was performed using an AB17700 sequence detection system (PE Applied Biosystems) in the presence of SYBRO-green. This fluorochrome incorporates stoichiometrically only in the double-stranded DNA. Primers for each gene of interest were designed for use under real-time PCR conditions (see FIG. 13), to amplify an 80-100 base pair fragment at 59° C. annealing temperature (Primers Express, PE Applied Biosystems). The optimisation of the real-time PCR reaction was then performed according to the manufacturer's instructions. For each triplicate analysis, transcription of the gene of interest was compared to transcription of the housekeeping gene GAPDH, which was amplified in parallel using conditions required by manufacturer's instructions.

The results, which are shown in FIGS. 11a and 11b, clearly show a marked increase in the expression IL-10 in CD4⁺ T-cells during the induction phase of tolerance, with peak levels of transcripts occuring two days after intranasal peptide treatment and returning to baseline levels by day 8.

### EXAMPLE 8

Maintenance of Notch Signalling During the Induction of Tolerance.

CD4⁺ cells were purified from the spleen of mice:

immunised with Der p 1/CFA (hatched bars); or

treated with Der p1, 110-131 intranasally on saline (closed bars).

Total RNA was isolted from the cells and reverse transcribed to cDNA and analysed by real time PCR to examine the expression of transcripts for Notch1, Notch2, Delta1, Serrate1, Serrate2 and Hes-1.

In more detail, total RNA was extracted from MACS purified CD4⁺ T cells using QIAGEN Rneasy Mini Kit (QIAGEN, UK). 400 ng of total RNA was reverse transcribed into cDNA using the TaqMan RT kit (PE Biosystems, UK). Briefly, a 20 μl reaction consisting of a final concentration of 1× TaqMan RT buffer, 5.5 mM magnesium chloride, 500 μM of each of dNTP, 2.5:M of random hexamers, 0.4 U/μl of RNAse inhibitor, 1.25 U/μl of MultiScribe RT and 400 ng of RNA, was incubated for 10 min at 25° C., 30 min at 48° and 5 min at 95° C. The cDNA was then used for real-time PCR. The method uses a fluorogenic probe that specifically anneals the template between the PCR primers. Suitable primers and probes are listed in FIG. 13. The use of a sequence detector (ABI Prism 7700, PE Biosystems) measures amplification of the product in direct proportion to the increase in fluorescence emitted by the probe. Each sample was run in duplicates in a 96-well optical reaction plate (PE Biosystems). 25 μl of the PCR reaction were added per well. The PCR reaction consisted of 2.5 μl of cDNA, 1.25 μl of RNAse/DNAse-free water, a final volume of 1× TaqMan Universal PCR Master Mix (PE Byosistems, UK), 1× Pre-Developed 18S rRNA, 7.5 pmol/μl of the forward and reverse primer and 5 pmol/μl of the probe The thermal cycler conditions were standard for all the genes tested. These were 2 min at 50° C., 10 min at 95° C. and 95° C. at 15 sec/60° C. at 1 min for 40 cycles. All the values obtained were normalised to 18S rRNA, which was included in the PCR reaction as an internal endogenous control. Furthermore, each sample was made relative to a relevant positive control, which always had a value of 1.

The results are shown in FIGS. 12A, B, C, D, E and F respectively and demonstrate that induction of T-cell tolerance modulates the expression of these genes. In particular, it was shown that Notch2, Serrate1, Serrate2 and Hes-1 expression was enhanced in tolerant mice.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

### REFERENCES

Lu, F. M. et al. (1996) Proc Natl Acad Sci 93:11 pp. 5663-7.

Lieber, T. et al. (1993) Genes Dev 7:10 pp. 1949-65.

Struhl, G. et al. (1998) Cell 93:4 pp. 649-60.

Ellisen, L. W. et al. (1991) Cell 66 pp. 649-661.

Robey, E. et al. (1996) Cell 87 pp. 483-492.

von Boehmer, H. et al. (1990) 8 p. 531.

Qin, S. X. et al. (1993) Science 253 p. 974.

Hoyne, G. F. et al. (1993) J Exp Med 178, p. 183.

Metzler, B. et al. (1993) nt Immunol 5, p.1159.

Matsuno, K. et al. (1995) Development 121(8), pp. 2633-44.

Schroeter, E. H. et al. (1998) Nature 393(6683):382-6.

Jarriault S. et al (1995) Nature 377(6547):355-8.

Hoyne G. F. et al (1999) Int Arch Allergy Immunol 118:122-124.

Hoyne G. F. et al (2000) Intl Immunol 12:177-185.

Hoyne G. F. et al (2000) Immunology 100:281-288.

Mullis and Faloona (1987) Meth Enzymol 55:335-350.

Takebayashi K. et al (1994) J Biol Chem 269(7):150-6.

Leimeister C. et al (1999) Mech Dev 85(1-2):173-7.

McGuinness T. et al (1996) Genomics 35(3):473-85.

Chee M. Et al (1996) Science 274:601-614.

Camilli et al (1994) Proc Natl Acad Sci USA 91:2634-2638.

Artavanis-Tsakomas et al. (1995) Science 268:225-232.

Artavanis-Tsakomas et al. (1999) Science 284:770-776.

Weinmaster (2000) Curr. Opin. Genet. Dev. 10:363-369.

Munro and Freeman (2000) Curr. Biol. 10:813-820.

Ju et al. (2000) Nature 405:191-195.

Moloney et al. (2000) Nature 406:369-375.

Brucker et al. (2000) Nature 406:411-415.

Panin et al. (1997) Nature 387:908-912.

Hicks et al. (2000) Nat. Cell. Biol. 2:515-520.

Irvine (1999) Curr. Opin. Genet. Devel. 9:434-441.

Devereux et al. (1984) Nucleic Acid Research 12:87.

Caux et al. (1992) Nature 360:258-261.

Coffin et al. (1998) Gene Therapy 5:718-722.

Osborne and Miele (1999) Immunity 11:653-663.

Ordentlich et al. (1998) Mol. Cell. Biol. 18:2230-2239.

Hampton R et al. (1990) Serological Methods, APS St Paul, Minn., USA.

Maddox D E et al. (1983) J Exp Med 15(8):1211.

Kohler and Milstein (1975), Nature 256:495-497.

Kozbor et al. (1983), Immunology Today 4:72.

Cole et al. (1985) Monoclonal Antibodies and Cancer Therapy, Alan R. Liss, Inc., pp. 77-96.

Huse et al. (1989) Science 246:1275-1281.

Meuer S. et al (2000) Rapid Cycle Real-time PCR, Springer-Verlag Berlin and Heidelberg GmbH & Co.

1. A method for monitoring the immune system comprising a step of detecting modulation of Notch signalling.

2. A method for monitoring the immune system comprising a step of detecting cleavage of the cleavable intracellular domain of Notch.

3. A method for monitoring the immune system comprising a step of detecting interaction of Suppresser of Hairless (Su(H)), or homologues thereof, with Notch.

4. A method for monitoring the immune system comprising a step of detecting interaction of Deltex, or homologues thereof, with Notch.

5. A method for monitoring the immune system comprising a step of detecting interaction of Deltex, or homologues thereof, with Grb2.

6. A method for monitoring the immune system comprising a step of detecting modulation of the Ras-JnK signalling pathway.

7. A method for monitoring the immune system comprising a step of detecting cleavage of the cleavable extracellular domain of Delta.

8. A method for detecting or monitoring T-cell activation or inactivation comprising a step of detecting modulation of Notch signalling.

9. A method for detecting or monitoring T-cell activation or inactivation comprising a step of detecting cleavage of the cleavable intracellular domain of Notch.

10. A method for detecting or monitoring T-cell activation or inactivation comprising a step of detecting interaction of Suppresser of Hairless (Su(H)), or homologues thereof, with Notch.

11. A method for detecting or monitoring T-cell activation or inactivation comprising a step of detecting interaction of Deltex, or homologues thereof, with Notch.

12. A method for detecting or monitoring T-cell activation or inactivation comprising a step of detecting interaction of Deltex, or homologues thereof, with Grb2.

13. A method for detecting or monitoring T-cell activation or inactivation comprising a step of detecting modulation of the Ras-JnK signalling pathway.

14. A method for detecting or monitoring T-cell activation or inactivation comprising a step of detecting cleavage of the cleavable extracellular domain of Delta.

15. A method for detecting or monitoring immunological tolerance or activity comprising a step of detecting modulation of Notch signalling.

16. A method for detecting or monitoring immunological tolerance or activity comprising a step of detecting cleavage of the cleavable intracellular domain of Notch.

17. A method for detecting or monitoring immunological tolerance or activity comprising a step of detecting interaction of Suppresser of Hairless (Su(H)), or homologues thereof, with Notch.

18. A method for detecting or monitoring immunological tolerance or activity comprising a step of detecting interaction of Deltex, or homologues thereof, with Notch.

19. A method for detecting or monitoring immunological tolerance or activity comprising a step of detecting interaction of Deltex, or homologues thereof, with Grb2.

20. A method for detecting or monitoring immunological tolerance or activity comprising a step of detecting modulation of the Ras-JnK signalling pathway.

21. A method for detecting or monitoring immunological tolerance or activity comprising a step of detecting cleavage of the cleavable extracellular domain of Delta.

22. A method for monitoring the efficacy of immunotherapy comprising a step of detecting modulation of Notch signalling.

23. A method for monitoring the efficacy of immunotherapy comprising a step of detecting cleavage of the cleavable intracellular domain of Notch.

24. A method for monitoring the efficacy of immunotherapy comprising a step of detecting interaction of Suppresser of Hairless (Su(H)), or homologues thereof, with Notch.

25. A method for monitoring the efficacy of immunotherapy comprising a step of detecting interaction of Deltex, or homologues thereof, with Notch.

26. A method for monitoring the efficacy of immunotherapy comprising a step of detecting interaction of Deltex, or homologues thereof, with Grb2.

27. A method for monitoring the efficacy of immunotherapy comprising a step of detecting modulation of the Ras-JnK signalling pathway.

28. A method for monitoring the efficacy of immunotherapy comprising a step of detecting cleavage of the cleavable extracellular domain of Delta.

29. A method for detecting or monitoring the reactivity of a T-cell to an antigen comprising a step of detecting modulation of Notch signalling.

30. A method for detecting or monitoring Notch signalling comprising a step of detecting modulation of Notch signalling.

31. A method for detecting or monitoring the reactivity of a T-cell to an antigen comprising a step of detecting cleavage of the cleavable intracellular domain of Notch.

32. A method for detecting or monitoring Notch signalling comprising a step of detecting cleavage of the cleavable intracellular domain of Notch.

33. A method for detecting or monitoring the reactivity of a T-cell to an antigen comprising a step of detecting interaction of Suppresser of Hairless (Su(H)), or homologues thereof, with Notch.

34. A method for detecting or monitoring Notch signalling comprising a step of detecting interaction of Suppresser of Hairless (Su(H)), or homologues thereof, with Notch.

35. A method for detecting or monitoring the reactivity of a T-cell to an antigen comprising a step of detecting interaction of Deltex, or homologues thereof, with Notch.

36. A method for detecting or monitoring Notch signalling comprising a step of detecting interaction of Deltex, or homologues thereof, with Notch.

37. A method for detecting or monitoring the reactivity of a T-cell to an antigen comprising a step of detecting interaction of Deltex, or homologues thereof, with Grb2.

38. A method for detecting or monitoring Notch signalling comprising a step of detecting interaction of Deltex, or homologues thereof, with Grb2.

39. A method for detecting or monitoring the reactivity of a T-cell to an antigen comprising a step of detecting modulation of the Ras-JnK signalling pathway.

40. A method for detecting or monitoring Notch signalling comprising a step of detecting modulation of the Ras-JnK signalling pathway.

41. A method for detecting or monitoring the reactivity of a T-cell to an antigen comprising the steps of:
a. providing a T-cell in the presence of an antigen;
b. detecting modulation of Notch signalling within the T-cell; and
c. determining the reactivity of the T-cell to said antigen.

42. A method according to claim 41 wherein step (b) comprises a step of detecting cleavage of the cleavable intracellular domain of Notch.

43. A method according to claim 41 wherein step (b) comprises a step of detecting interaction of Suppressor of Hairless (Su(H)), or homologues thereof, with Notch.

44. A method according to claim 41 wherein step (b) comprises a step of detecting interaction of Deltex, or homologues thereof, with Notch.

45. A method according to claim 41 wherein step (b) comprises a step of detecting interaction of Deltex, or homologues thereof, with Grb2.

46. A method according to claim 41 wherein step (b) comprises a step of detecting modulation of the Ras-JnK signalling pathway.

47. A method according to claim 41 wherein step (b) comprises a step of detecting cleavage of the cleavable extracellular domain of Delta.

48. A method according to any preceding claim further comprising a step of comparing detectable Notch signalling with a reference signal.

49. A method according to any preceding claim wherein modulation of Notch signalling is detected using a nucleic acid assay.

50. A method according to any of claims 1-48 wherein modulation of Notch signalling is detected using a protein assay.

51. A method according to claim 50 comprising the step of detecting a protein or protein fragment selected from the group consisting of: cleaved Notch intracellular domain, cleaved Notch extracellular domain and cleaved Delta extracellular domain.

52. A method according to any preceding claim comprising a step of detecting modulation of the expression of a target gene of Notch signalling.

53. A method according to claim 27 wherein the target gene is selected from any one or more of Deltex, Suppressor of Deltex, genes of the Hes family including Hes-1, E(spl) complex genes, cytokine genes including Il-10, CD antigen genes including CD-4 and CD-23, CTLA-4, Dll-1, Numb, genes of the Fringe family, Mastermind, Dsh, dlx-1, Su(H), CBF1, genes of the Delta family including Delta 1-4, genes of the Serrate family including Serrate 1 and 2 and genes of the Notch family including Notch 1-4, genes encoding members of the Ras-Jnk pathway, homologues, variants, fragments and derivatives thereof.

54. A method according to claim 53 wherein the target gene is not IL-10.

55. A method according to claim 53 wherein the target gene is not a cytokine.

56. A method according to claim 52 wherein the target gene is a synthetic reporter gene.

57. A method according to claim 56 wherein the target gene is any one or more of a gene encoding a polypeptide having an enzymatic activity, a gene comprising a radiolabel or a fluorescent label and a gene encoding a predetermined polypeptide epitope.

58. A method for detecting or monitoring the reactivity of a T-cell to an antigen comprising the steps of:
a. providing a ligand capable of binding to Notch such as to modulate Notch signalling;
b. providing a T-cell having a Notch receptor or a functional variant thereof capable of transducing Notch signalling;
c. allowing the ligand to bind to Notch in the presence of an antigen;
d. detecting modulation of Notch signalling; and
e. determining the reactivity of the T-cell to said antigen using a method according to any preceding claim.

59. A method for detecting or monitoring Notch signalling comprising the steps of:
a. providing a ligand capable of binding to Notch such as to modulate Notch signalling;
b. providing a Notch receptor or a functional variant thereof;
c. allowing the ligand to bind to Notch; and
d. detecting modulation of Notch signalling using a method according to any of claims 1-57.

60. A method according to any preceding claim for detecting increased reactivity of a T-cell to an antigen.

61. A method according to any of claims 1-59 for detecting decreased reactivity of a T-cell to an antigen.

62. A method according to claim 61 for detecting tolerisation of a T-cell to an antigen.

63. A method according to any preceding claim wherein the antigen is a self antigen.

64. A method according to any of claims 1-62 wherein the antigen is a foreign antigen.

65. A kit for detecting or monitoring modulation of Notch signalling comprising a plurality of sequences or binding agents specific to nucleic acid or protein targets of Notch signalling.

66. A kit according to claim 65 which is substantially free of unrelated sequences or binding agents.

67. A nucleic acid or protein array consisting essentially of a plurality of sequences or binding agents specific to nucleic acid or protein targets of Notch signalling.

68. A method of screening for modulators of Notch signalling comprising the steps of:
a. providing a candidate modulator;
b. providing a Notch receptor or a functional variant thereof;
c. bringing Notch and the candidate modulator together under conditions such that the modulator interacts with Notch; and
d. detecting modulation of Notch signalling using a method according to any one of claims 1-67.

69. A modulator of Notch signalling identifiable using the method of claim 68.

70. The use of a detectable signal of Notch signalling for detecting or monitoring the reactivity of a T-cell to an antigen.

71. The use of a detectable signal of Notch signalling for monitoring the immune system.

72. The use of a detectable signal of Notch signalling for detecting or monitoring T-cell activation or inactivation.

73. The use of a detectable signal of Notch signalling for detecting or monitoring immunological tolerance or activation.
